(19) United States
(12) Patent Application Publication
(10) Pub. No.: US 2007/0261138 A1
(43) Pub. Date: Nov. 8, 2007

(54) Synthetase Enzymes

(76) Inventors: Ian Alexander Graham, York (GB); Thierry Tonon, Roscoff (FR)

Correspondence Address: KLARQUIST SPARKMAN, LLP, 121 SW SALMON STREET, SUITE 1600, PORTLAND, OR, 97204, US

(21) Appl. No.: 11/574,219

(22) PCT Filed: Sep. 21, 2005
(86) PCT No.: PCT/GB05/03643

§ 371 Date: Feb. 23, 2007

(30) Foreign Application Priority Data
Oct. 2, 2004 (GB) 0421937.4

(51) Int. Cl. A01H 9/00; C07K 14/00; C09F 7/00; C12N 1/00; C12N 15/00; C12N 5/00

(52) U.S. Cl. 800/295; 435/257.2; 435/320.1; 435/410; 530/350; 554/30

(57) ABSTRACT

We describe transgenic cells expressing algal acyl-CoA synthetases and including processes to esterify long chain fatty acids with coenzyme A

The invention relates to transgenic cells expressing algal acyl Co-A synthetases.

Cellular storage of fatty acids in triacylglycerol requires that the fatty acids are first activated to their acyl-CoA esters through the action of acyl-CoA synthetase enzymes. Acyl-CoAs are produced by acyl-CoA synthetase from fatty acid, ATP and Coenzyme A. Acyl-CoA synthetases can exhibit substrate specificity for different chain length or different degrees of saturation of the fatty acid. For example an arachidonate (20:4n-6)-preferring acyl-CoA synthetase has been identified in rat. This enzyme has a high affinity for arachidonate and eicosapentaenoic acid (EPA) and low affinity for palmitate. Several isoforms of acyl-CoA synthetases have also been identified in Arabidopsis. Acyl-CoA synthetases (ACSs) play a critical role in the biosynthetic pathways of nearly all fatty acid-derived molecules. Long chain acyl CoA synthetase (LACS) enzymes esterifies free fatty acids to coenzyme A to form acyl CoAs, a key activation step that is necessary for the utilization of fatty acids by most lipid metabolic enzymes [1].

The enzymatic mechanism is a two-step reaction that proceeds via the formation of an acyl-adenylate (acyl-AMP) intermediate [2]. Acyl-CoAs serve as important intermediates in many metabolic pathways, such as elongation and β-oxidation of fatty acids, enzyme activation, cell signalling, and transcriptional regulation [3]. Consistent with the diverse roles of acyl-CoA synthetases (ACS) in cell metabolism, many eukaryotic organisms encode several different ACSs that specifically activate short (C6-C8), medium (C10-C12), long (C14-C20), or very long (>C22) chain-length fatty acids [3]. Moreover, some organisms possess multiple enzymes for each set of acyl chain lengths. In plants, LACS activity has been localized to several sub-cellular compartments [4,5], enabling acyl chains produced by de novo fatty acid synthesis to be activated to their CoA esters and subsequently used for metabolic pathways such as those involved in the synthesis of membrane glycerolipids and storage lipids (triacylglycerols, TAGs) in developing seeds [6].

In addition, LACS enzymes play an important role in fatty acid transport. This process has been studied in detail in bacteria [7], yeast (Saccharornyces cerevisiae) [8], and mammalian cells [9].

Marine microalgae produce a wide variety of fatty acids, and some species have attracted interest because they contain health beneficial polyunsaturated fatty acids (PUFAs) [11]. Herein below, polyunsaturated fatty acids are referred to as PUFA, PUFAs, LCPUFA or LCPUFAs (poly unsaturated fatty acids, PUFA, long chain poly unsaturated fatty acids, LCPUFA). The ultimate reconstruction of the microalgal very long chain polyunsaturated fatty acids (VLCPUFA) biosynthetic pathway in higher plants is a desirable goal, but will require the introduction of multiple enzymatic reactions including fatty acid desaturation, elongation, and activation to form substrates suitable for incorporation into TAGs.

In our co-pending applications we describe nucleic acid molecules encoding activities associated with PUFA biosynthetic pathways. In WO03/078639, which is incorporated by reference (in particular the nucleic acid sequences therein disclosed), we describe several enzyme activities, for example elongases, desaturases, acyl-CoA synthetases and diacylglycerol acyltransferases that are involved in the modification of long chain fatty acids. These nucleic acid molecules are isolated from the algal species Pavlova lutheri. In our currently unpublished application PCT/GB04/003057, which is incorporated by reference (in particular the nucleic acid sequences therein disclosed), we describe the characterisation of elongase polypeptides isolated from the algal species Thalassiosira pseudonana. Furthermore, we describe in our currently unpublished application GB0403452.6, which is incorporated by reference (in particular the nucleic acid sequences therein disclosed), enzymes with novel desaturase activity. For example, a cytochrome b5 desaturase exhibiting Δ11-desaturase activity and a further enzyme that has Δ6-desaturase activity, each of which are isolated from Thalassiosira pseudonana.

We describe the characterization of an acyl-CoA synthetase (TplascA) gene of Thalassiosira pseudonana. This enzyme exhibits high activity towards the health beneficial VLCPUFAs EPA and docosahexaenoic acid (DHA), and has been shown to increase the quantity of DHA stored in yeast TAGs.

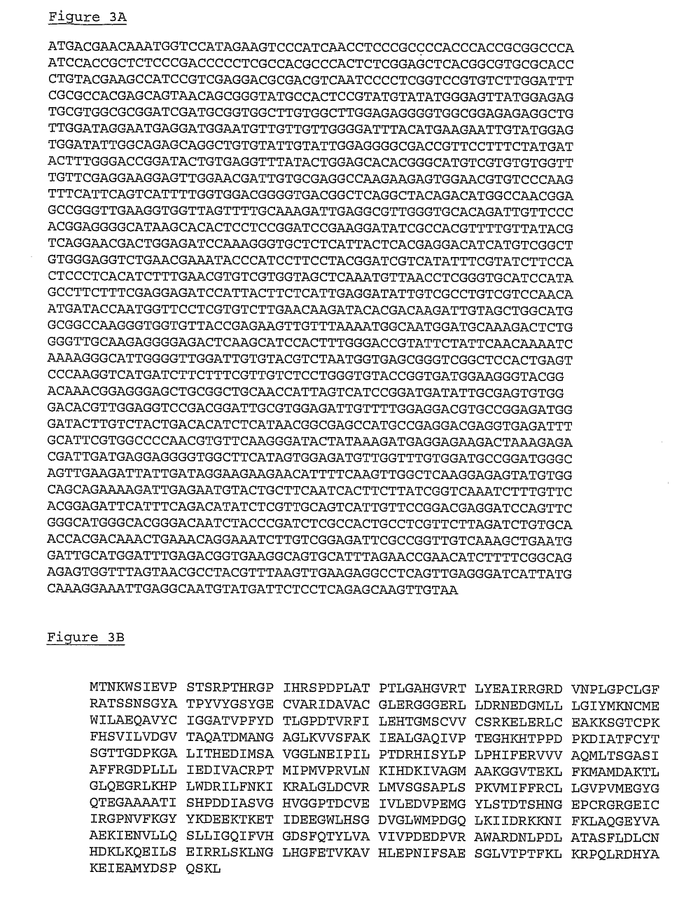

According to an aspect of the invention there is provided a transgenic cell comprising a nucleic acid molecule which comprises a nucleic acid sequence which nucleic acid molecule consists of the sequence as represented in FIG. 3A, or nucleic acid molecules that hybridize to this sequence under stringent hybridization conditions, wherein said nucleic acid molecule encodes a polypeptide which has acyl co A synthetase activity.

In a preferred embodiment of the invention said nucleic acid molecule comprises a nucleic acid sequence which has about 50% homology to the nucleic acid sequence represented in FIG. 3A.

Preferably said homology is at least 50%, 60%, 70%, 80%, 90%, or at least 99% identity with the nucleic acid sequence represented in FIG. 3A and which encodes a polypeptide which has acyl-CoA synthetase activity.

In a preferred embodiment of the invention said nucleic acid molecule comprises the nucleic acid sequence as represented in FIG. 3A. Preferably said nucleic acid molecule consists of the nucleic acid sequence as represented in FIG. 3A.

According to a further aspect of the invention there is provided a transgenic cell wherein said cell is adapted to express a nucleic acid molecule that encodes a polypeptide as represented by the amino acid sequence shown in FIG. 3B, or a variant amino acid sequence which sequence is modified by addition, deletion or substitution of at least one amino acid residue and wherein said polypeptide, or variant polypeptide has acyl-CoA synthetase activity.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (allows sequences that share
at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share at least 80% identity
to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share at least 50% identity
to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said modification retains or enhances the enzyme activity of said polypeptide.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a preferred embodiment of the invention said nucleic acid molecules are isolated from an algal species.

Preferably said algal species is selected from the group consisting of: Amphidinium carterae, Amphiphora hyalina, Amphiphora sp., Chaetoceros gracilis, Coscinodiscus sp., Crypthecodinium cohnii, Cryptomonas sp., Cylindrotheca fusiformis, Haslea ostrearia, Isochrysis galbana, Nannochloropsis oculata, Navicula sp., Nitzschia closterium, Pavlova lutheri, Phaeodactylum tricornutum, Prorocentrum minimum, Rhizosolenia setigera, Skeletonema costatum, Skeletoneina sp., Tetraselmis tetrathele, Thalassiosira nitzschioides, Thalassiosira heterophorma, Thalassiosira pseudonana, Thalassiosira stellaris.

In a preferred embodiment of the invention said acyl-CoA synthetase activity modifies 20 and/or 22 carbon polyunsaturated fatty acids. Preferably said fatty acids are 20:4n6, 20:5n3 or 22:6n3 carbon polyunsaturated fatty acids.

According to a further aspect of the invention there is provided a vector comprising the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection.

Preferably the nucleic acid in the vector is operably linked to an appropriate promoter or other regulatory elements for transcription in a host cell such as a prokaryotic, (e.g. bacterial), or eukaryotic (e.g. fungal, plant, mammalian or insect cell). The vector may be a bi-functional expression vector which functions in multiple hosts. In the example of nucleic acids encoding polypeptides according to the invention this may contain its native promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al (1985) Nature 313, 9810-812); rice actin (McElroy et al (1990) Plant Cell 2: 163-171); ubiquitin (Christian et al. (1989) Plant Mol. Biol. 18 (675-689); pEMU (Last et al (1991) Theor Appl. Genet. 81: 581-588); MAS (Velten et al (1984) EMBO J. 3. 2723-2730); ALS promoter (U.S. application Ser. No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induced gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al (1991) Proc. Natl. Acad. Sci. USA 88: 10421-10425 and McNellie et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference.

Where enhanced expression in particular tissues is desired, tissue-specific promoters can be utilised. Tissue-specific promoters include those described by Yamamoto et al. (1997) Plant J. 12(2): 255-265; Kawamata et al (1997) Plant Cell Physiol. 38(7): 792-803; Hansen et al (1997) Mol. Gen. Genet. 254(3): 337-343; Russell et al. (1997) Transgenic Res. 6(2): 157-168; Rinehart et al (1996) Plant Physiol. 112(3): 1331-1341; Van Camp et al (1996) Plant Physiol. 112(2): 525-535; Canevascni et al (1996) Plant Physiol. 112(2): 513-524; Yamamoto et al (1994) Plant Cell Physiol. 35(5): 773-778; Lam (1994) Results Probl. Cell Differ. 20: 181-196; Orozco et al (1993) Plant Mol. Biol. 23(6): 1129-1138; Mutsuoka et al (1993) Proc. Natl. Acad. Sci. USA 90(20): 9586-9590; and Guevara-Garcia et al (1993) Plant J. 4(3): 495-50.

In a preferred embodiment of the invention said tissue specific promoter is a promoter which is active during the accumulation of oil in developing oil seeds; see Broun et al. (1998) Plant J. 13(2): 201-210.

“Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is an inducible promoter or a developmentally regulated promoter.

Particular vectors are nucleic acid constructs which operate as plant vectors. Specific procedures and vectors previously used with wide success upon plants are described by Guerineau and Mullineaux (1993) (Plant transformation and expression vectors.

In: Plant Molecular Biology Labfax (Croy RRD ed) Oxford, BIOS Scientific Publishers, pp 121-148. Suitable vectors may include plant viral-derived vectors (see e.g. EP-A-194809).

Vectors may also include selectable genetic marker such as those that confer selectable phenotypes such as resistance to herbicides (e.g. kanamycin, hygromycin, phosphinotricin, chlorsulfuron, methotrexate, gentamycin, spectinomycin, imidazolinones and glyphosate).

Alternatively, or in addition, said vectors are vectors suitable for mammalian cell transfection or yeast cell transfection. In the latter example multi-copy vectors such as 2μ episomal vectors are preferred. Alternatively yeast CEN vectors and intergrating vectors such as YIP vectors are suitable for transformation of yeast species such as Saccharomyces cerevisiae and Pichia spp.

In a further preferred embodiment of the invention said cell over-expresses the encoded by said nucleic acid molecule.

In a preferred embodiment of the invention said over-expression is at least 2-fold higher when compared to a non-transformed reference cell of the same species.

Preferably said over-expression is: at least 3-fold, 4-fold, 5-fold, 6-fold, 7-fold, 8-fold, 9-fold, or at least 10-fold when compared to a non-transformed reference cell of the same species.

In a preferred embodiment of the invention said nucleic acid molecule is a cDNA.

In yet a further preferred embodiment of the invention said nucleic acid molecule is a genomic DNA.

In a preferred embodiment of the invention said transgenic cell is a eukaryotic cell.

In an alternative preferred embodiment of the invention said cell is a prokaryotic cell.

In a further preferred embodiment of the invention said eukaryotic cell is a plant cell.

Plants which include a plant cell according to the invention are also provided as are seeds produced by said plants.

In a preferred embodiment of the invention said plant is selected from: corn (Zea mays), canola (Brassica napus, Brassica rapa ssp.), flax (Linum usitatissimum), alfalfa (Medicago sativa), rice (Oryza sativa), rye (Secale cerale), sorghum (Sorghum bicolor, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plants of the present invention are crop plants (for example, cereals and pulses, maize, wheat, potatoes, tapioca, rice, sorghum, millet, cassava, barley, pea), and other root, tuber or seed crops. Important seed crops are oil-seed rape, sugar beet, maize, sunflower, soybean, sorghum, and flax (linseed). Horticultural plants to which the present invention may be applied may include lettuce, endive, and vegetable brassicas including cabbage, broccoli, and cauliflower. The present invention may be applied in tobacco, cucurbits, carrot, strawberry, sunflower, tomato, pepper.

Grain plants that provide seeds of interest include oil-seed plants and leguminous plants. Seeds of interest include grain seeds, such as corn, wheat, barley, rice, sorghum, rye, etc.

Oil seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chickpea, etc.

According to a further aspect of the invention there is provided a seed comprising a plant cell according to the invention. Preferably said seed is from an oil seed plant.

According to an aspect of the invention there is provided the use of a polypeptide or cell according to the invention in the esterification of a long chain fatty acid to coenzyme A to form acyl-CoA.

According to a yet further aspect of the invention there is provided a reaction vessel comprising a polypeptide according to the invention, long chain fatty acid, ATP and coenzyme A. Preferably said vessel is a fermentor.

In a preferred embodiment of the invention said polypeptide is expressed by a cell according to the invention.

Preferably said cell is a eukaryotic cell, for example a yeast cell.

In an alternative preferred embodiment of the invention said cell is a prokaryotic cell.

According to a further aspect of the invention there is provided a process to esterify a long chain fatty acid substrate to coenzyme A to form acyl-CoA comprising the steps of:
i) providing a reaction vessel according to the invention; and
ii) growing cells contained in said reaction vessel under conditions which allow the esterification of a long chain fatty acid to acyl-CoA.

Advantageously, the polyunsaturated fatty acids produced in the process of the invention comprise at least two, advantageously three, four or five, double bonds. The fatty acids particularly advantageously comprise four or five double bonds. Fatty acids produced in the process advantageously have 18, 20, 22 or 24 carbon atoms in the fatty acid chain; preferably, the fatty acids comprise 20, 22 or 24 carbon atoms in the fatty acid chain. Advantageously, saturated fatty acids are reacted to a minor extent, or not at all, with the nucleic acids used in the process. A minor extent is understood as meaning that the saturated fatty acids are reacted with less than 5%, advantageously less than 3%, especially advantageously with less than 2% of the activity in comparison with polyunsaturated fatty acids. These fatty acids which are produced may be produced in the process as a single product or be present in a fatty acid mixture.

In a preferred method of the invention said long chain fatty acid is selected from the group consisting of: 18:3n6, 20:4n6, 18:4n3, 20:5n3 and 22:6n3.

The polyunsaturated fatty acids produced in the process are advantageously bound in membrane lipids and/or triacylglycerides but may also occur in the organisms as free fatty acids or else bound in the form of other fatty acid esters. In this context, they may be present as stated as “pure products” or else advantageously in the form of mixtures of various fatty acids or mixtures of different glycerides. The various fatty acids bound in the triacylglycerides can be derived here from short-chain fatty acids having from 4 to 6 carbon atoms, medium-chain fatty acids having from 8 to 12 carbon atoms or long-chain fatty acids having from 14 to 24 carbon atoms, with preference being given to the long-chain fatty acids and particular preference being given to the long-chain fatty acids, LCPUFAs, of C₁₈-, C₂₀-, C₂₂- and/or C₂₄-fatty acids.

The process of the invention advantageously produces fatty acid esters with polyunsaturated C₁₈-, C₂₀-, C₂₂- and/or C₂₄-fatty acid molecules, with at least two double bonds being present in the fatty acid ester. These fatty acid molecules preferably comprise three, four or five double bonds and advantageously lead to the synthesis of hexadecadienoic acid (C16:2^{Δ9,12}), γ-linolenic acid (=GLA, C18:3^{Δ6,9,12}), stearidonic acid (=SDA, C18:4^{Δ6,9,12,15}), dihomo-γ-linolenic acid (=DGLA, 20:3^{Δ8,11,14}), eicosatetraenoic acid (=ETA, C20:4^{Δ}5,8,11,14), arachidonic acid (ARA), eicosapentaenoic acid (EPA) or mixtures thereof, preferably EPA and/or ARA.

The fatty acid esters with polyunsaturated C₁₈-, C₂₀-, C₂₂- and/or C₂₄-fatty acid molecules can be isolated, from the organisms which have been used for the preparation of the fatty acid esters, in the form of an oil or lipid, for example in the form of compounds such as sphingolipids, phosphoglycerides, lipids, glycolipids such as glycosphingolipid, phospholipids such as phosphatidylethanolamine, phosphatidylcholine, phosphatidylserine, phosphatidylglycerol, phosphatidylinositol or diphosphatidylglycerol, monoacylglycerides, diacylglycerides, triacylglycerides which comprise the polyunsaturated fatty acids with at least two, preferably three double bonds; advantageously they are isolated in the form of their diacylglycerides, triacylglycerides and/or in the form of phosphatidylcholine, especially preferably in the form of the triacylglycerides. In addition to these esters, the polyunsaturated fatty acids are also present in the organisms, advantageously the plants, as free fatty acids or bound in other compounds. As a rule, the various abovementioned compounds (fatty acid esters and free fatty acids) are present in the organisms with an approximate distribution of 80 to 90% by weight of triglycerides, 2 to 5% by weight of diglycerides, 5 to 10% by weight of monoglycerides, 1 to 5% by weight of free fatty acids, 2 to 8% by weight of phospholipids, the total of the various compounds amounting to 100% by weight.

The process according to the invention yields the LCPUFAs produced in a content of at least 3% by weight, advantageously at least 5% by weight, preferably at least 8% by weight, especially preferably at least 10% by weight, most preferably at least 15% by weight, based on the total fatty acids in the transgenic organisms, advantageously in a transgenic plant. The fatty acids are advantageously produced in bound form. With the aid of the nucleic acids used in the process according to the invention, these unsaturated fatty acids can be brought into the sn1, sn2 and/or sn3 position of the triglycerides which are advantageously prepared. Since a plurality of reaction steps are performed by the starting compounds hexadecadienoic acid (C16:2), linoleic acid (C18:2) and linolenic acid (C18:3) in the process according to the invention, the end products of the process such as, for example, arachidonic acid (ARA) or eicosapentaenoic acid (EPA) or docosahexaenoic acid (DHA), are not obtained as absolutely pure products; minor traces of the precursors are always present in the end product. If, for example, both linoleic acid and linolenic acid are present in the starting organism and the starting plant, the end products such as ARA and EPA are present as mixtures. The precursors should advantageously not amount to more than 20% by weight, preferably not to more than 15% by weight, especially preferably not to more than 10% by weight, most preferably not to more than 5% by weight, based on the amount of the end product in question. Advantageously, only ARA or only EPA, bound or as free acids, are produced as end products in a transgenic plant in the process according to the invention. If both compounds (ARA and EPA) are produced simultaneously, they are advantageously produced in a ratio of at least 1:2 (EPA:ARA), advantageously of at least 1:3, preferably 1:4, especially preferably 1:5.

Owing to the nucleic acid sequences according to the invention, an increase in the yield of polyunsaturated fatty acids of at least 50%, advantageously of at least 80%, especially advantageously of at least 100%, very especially advantageously of at least 150%, in comparison with the non-transgenic starting organism, can be obtained by comparison in GC analysis. In a further advantageous embodiment, the yield of polyunsaturated fatty acids can be increased by at least 200%, preferably by at least 250%, very especially preferably by at least 300%.

Chemically pure polyunsaturated fatty acids or fatty acid compositions can also be synthesized by the processes described above. To this end, the fatty acids or the fatty acid compositions are isolated from the organism, such as the microorganisms or the plants or the culture medium in or on which the organisms have been grown, or from the organism and the culture medium, in the known manner, for example via extraction, distillation, crystallization, chromatography or combinations of these methods. These chemically pure fatty acids or fatty acid compositions are advantageous for applications in the food industry sector, the cosmetics industry sector and especially the pharmacological industry sector.

Suitable organisms for the production in the process according to the invention are, in principle, any organisms such as microorganisms, non-human animals or plants. Advantageously the process according to the invention employs transgenic organisms such as fungi, such as Mortierella or Traustochytrium, yeasts such as Saccharomyces or Schizosaccharomyces, mosses such as Physcomitrella or Ceratodon, non-human animals such as Caenorhabditis, algae such as Crypthecodinium or Phaeodactylum or plants such as dicotyledonous or monocotyledonous plants. Organisms which are especially advantageously used in the process according to the invention are organisms which belong to the oil-producing organisms, that is to say which are used for the production of oils, such as fungi, such as Mortierella or Traustochytrium, algae such as Crypthecodinium, Phaeodactylum, or plants, in particular plants, preferably oil crop plants which comprise large amounts of lipid compounds, such as peanut, oilseed rape, canola, sunflower, safflower, poppy, mustard, hemp, castor-oil plant, olive, sesame, Calendula, Punica, evening primrose, verbascum, thistle, wild roses, hazelnut, almond, macadamia, avocado, bay, pumpkin/squash, linseed, soybean, pistachios, borage, trees (oil palm, coconut or walnut) or arable crops such as maize, wheat, rye, oats, triticale, rice, barley, cotton, cassaya, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut). Especially preferred are plants which are high in C18:2- and/or C18:3-fatty acids, such as sunflower, safflower, tobacco, verbascum, sesame, cotton, pumpkin/squash, poppy, evening primrose, walnut, linseed, hemp, thistle or safflower. Very especially preferred plants are plants such as safflower, sunflower, poppy, evening primrose, walnut, linseed or hemp.

It is advantageous to the inventive process described to introduce, in addition to the nucleic acids according to the invention, further nucleic acids which code for enzymes of the fatty acid or lipid metabolism into the organism.

In principle, all genes of the fatty acid or lipid metabolism can be used in the process for the production of polyunsaturated fatty acids, advantageously in combination with the inventive acyl co A synthetase. Genes of the fatty acid or lipid metabolism selected from the group consisting of: acyl-CoA:lysophospholipid acyltransferase, acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferases, fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenases, triacylglycerol lipases, alleneoxide synthases, hydroperoxide lyases or fatty acid elongase(s) are advantageously used in combination with the acyl co A synthetase. Genes selected from the group of the acyl-CoA:lysophospholipid acyltransferases, Δ-4-desaturases, Δ-5-desaturases, Δ-6-desaturases, Δ-8-desaturases, Δ-9-desaturases, Δ-12-desaturases, Δ-5-elongases, Δ-6-elongases or Δ-9-elongases are especially preferably used in combination with the abovementioned genes for acyl co A synthetase, glycerol-3-phosphate acyltransferase, diacylglycerol acyltransferase or lecithin cholesterol acyltransferase, it being possible to use individual genes or a plurality of genes in combination.

Owing to the enzymatic activity of the nucleic acids used in the process according to the invention which code for polypeptides with lysophosphatidic acid acyltransferase glycerol-3-phosphate acyltransferase, diacylglycerol acyltransferase or lecithin cholesterol acyltransferase activity, advantageously in combination with nucleic acid sequences which code for polypeptides of the fatty acid or lipid metabolism, such as the acyl co A synthetase, the Δ-4-, Δ-5-, Δ-6-, Δ-8-desaturase or the Δ-5-, Δ-6- or Δ-9-elongase activity, a wide range of polyunsaturated fatty acids can be produced in the process according to the invention. Depending on the choice of the organisms, such as the advantageous plant, used for the process according to the invention, mixtures of the various polyunsaturated fatty acids or individual polyunsaturated fatty acids, such as EPA or ARA or DHA, can be produced in free or bound form. Depending on the prevailing fatty acid composition in the starting plant (C18:2- or C18:3-fatty acids), fatty acids which are derived from C18:2-fatty acids, such as GLA, DGLA or ARA, or fatty acids which are derived from C18:3-fatty acids, such as SDA, ETA, EPA or DHA, are thus obtained. If only linoleic acid (=LA, C18:2^{Δ9,12}) is present as unsaturated fatty acid in the plant used for the process, the process can only afford GLA, DGLA and ARA as products, all of which can be present as free fatty acids or in bound form. If only α-linolenic acid (=ALA, C18:3^{Δ9,12,15}) is present as unsaturated fatty acid in the plant used for the process, as is the case, for example, in linseed, the process can only afford SDA, ETA and EPA as products, all of which can be present as free fatty acids or in bound form, as described above. By modifying the activity of the enzymes involved in the synthesis, lysophosphatidic acid acyltransferase, glycerol-3-phosphate acyltransferase, diacylglycerol acyltransferase or lecithin cholesterol acyltransferase advantageously in combination with acyl co A synthetase, Δ-5-, Δ-6-desaturase and/or Δ-6-elongase or with acyl co A synthetase, Δ-5-, Δ-8-desaturase and/or Δ-9-elongase or in combination with only the first three genes, acyl co A synthetase, Δ-6-desaturase and/or Δ-6-elongase, acyl co A synthetase, Δ-8-desaturase and Δ-9-elongase, of the synthesis cascade, it is possible to produce, in a targeted fashion, only individual products in the abovementioned organisms, advantageously in the above-mentioned plants. Owing to the activity of Δ-6-desaturase and Δ-6-elongase, for example, GLA and DGLA, or SDA and ETA, are formed, depending on the starting plant and unsaturated fatty acid. DGLA or ETA or mixtures of these are preferably formed. If Δ-5-desaturase is additionally introduced into the organisms, advantageously into the plant, ARA or EPA is additionally formed. This also applies to organisms into which Δ-8-desaturase and Δ-9-elongase have been introduced previously. Advantageously, only ARA or EPA or mixtures of these are synthesized, depending on the fatty acid present in the organism, or in the plant, which acts as starting substance for the synthesis. Since biosynthetic cascades are involved, the end products in question are not present in pure form in the organisms. Small amounts of the precursor compounds are always additionally present in the end product. These small amounts amount to less than 20% by weight, advantageously less than 15% by weight, especially advantageously less than 10% by weight, most advantageously less than 5, 4, 3, 2 or 1% by weight, based on the end product DGLA, ETA or their mixtures, or ARA, EPA, DHA or their mixtures.

To increase the yield in the described method for the production of oils and/or triglycerides with an advantageously elevated content of polyunsaturated fatty acids, it is advantageous to increase the amount of starting product for the synthesis of fatty acids; this can be achieved for example by introducing, into the organism, a nucleic acid which codes for a polypeptide with Δ-12-desaturase. This is particularly advantageous in oil-producing organisms such as oilseed rape which are high in oleic acid. Since these organisms are only low in linoleic acid (Mikoklajczak et al., Journal of the American Oil Chemical Society, 38, 1961, 678-681), the use of the abovementioned Δ-12-desaturases for producing the starting material linoleic acid is advantageous.

Nucleic acids used in the process according to the invention are advantageously derived from plants such as algae such as Isochrysis or Crypthecodinium, algae/diatoms such as Phaeodactylum, mosses such as Physcomitrella or Ceratodon, or higher plants such as the Primulaceae such as Aleuritia, Calendula stellata, Osteospermum spinescens or Osteospermum hyoseroides, microorganisms such as fungi, such as Aspergillus, Thraustochytrium, Phytophthora, Entomophthora, Mucor or Mortierella, bacteria such as Shewanella, yeasts or animals such as nematodes such as Caenorhabditis, insects or humans. The nucleic acids are advantageously derived from fungi, animals, or from plants such as algae or mosses, preferably from nematodes such as Caenorhabditis.

The process according to the invention advantageously employs the abovementioned nucleic acid sequences or their derivative or homologs which code for polypeptides which retain the enzymatic activity of the proteins encoded by nucleic acid sequences. These sequences in combination with the nucleic acid sequences which code for acyl-CoA synthetase are cloned into expression constructs and used for the introduction into, and expression in, organisms. Owing to their construction, these expression constructs make possible an advantageous optimal synthesis of the polyunsaturated fatty acids produced in the process according to the invention.

In a preferred embodiment, the process furthermore comprises the step of obtaining a cell or an intact organism which comprises the nucleic acid sequences used in the process, where the cell and/or the organism is transformed with a nucleic acid sequence according to the invention, a gene construct or a vector as described below, alone or in combination with further nucleic acid sequences which code for proteins of the fatty acid or lipid metabolism. In a further preferred embodiment, this process furthermore comprises the step of obtaining the fine chemical from the culture. The culture can, for example, take the form of a fermentation culture, for example in the case of the cultivation of microorganisms, such as, for example, Mortierella, Saccharomyces or Traustochytrium, or a greenhouse- or field-grown culture of a plant. The cell or the organism produced thus is advantageously a cell of an oil-producing organism, such as an oil crop plant, such as, for example, peanut, oilseed rape, canola, linseed, hemp, soybean, safflower, sunflowers or borage.

In the case of plant cells, plant tissue or plant organs, “growing” is understood as meaning, for example, the cultivation on or in a nutrient medium, or of the intact plant on or in a substrate, for example in a hydroponic culture, potting compost or on arable land.

For the purposes of the invention, “transgenic” or “recombinant” means, with regard to the example of a nucleic acid sequence, an expression cassette (=gene construct) or a vector comprising the nucleic acid sequence according to the invention or an organism transformed with the nucleic acid sequences, expression cassette or vector according to the invention, all those constructions brought about by recombinant methods in which either;
a) the nucleic acid sequence according to the invention, or
b) a genetic control sequence which is operably linked with the nucleic acid sequence according to the invention, for example a promoter, or
c) (a) and (b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original organism or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the inventive nucleic acid sequences becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic organism or transgenic plant for the purposes of the invention is understood as meaning, as above, that the nucleic acids used in the process are not at their natural locus in the genome of an organism, it being possible for the nucleic acids to be expressed homologously or heterologously. However, as mentioned, transgenic also means that, while the nucleic acids according to the invention are at their natural position in the genome of an organism, the sequence has been modified with regard to the natural sequence, and/or that the regulatory sequences of the natural sequences have been modified. Transgenic is preferably understood as meaning the expression of the nucleic acids according to the invention at an unnatural locus in the genome, i.e. homologous or, preferably, heterologous expression of the nucleic acids takes place. Preferred transgenic organisms are fungi such as Mortierella, mosses such as Physcomitrella, algae such as Cryptocodinium or plants such as the oil crop plants.

Suitable organisms or host organisms for the nucleic acids, the expression cassette or the vector used in the process according to the invention are, in principle, advantageously all organisms which are capable of synthesizing fatty acids, specifically unsaturated fatty acids, and/or which are suitable for the expression of recombinant genes. Examples which may be mentioned are plants such as Arabidopsis, Asteraceae such as Calendula or crop plants such as soybean, peanut, castor-oil plant, sunflower, maize, cotton, flax, oilseed rape, coconut, oil palm, safflower (Carthamus tinctorius) or cacao bean, microorganisms, such as fingi, for example the genus Mortierella, Thraustochytrium, Saprolegnia, or Pythium, bacteria, such as the genus Escherichia, or Shewanella, yeasts, such as the genus Saccharomyces, cyanobacteria, ciliates, algae or protozoans such as dinoflagellates, such as Crypthecodinium. Preferred organisms are those which are naturally capable of synthesizing substantial amounts of oil, such as fungi, such as Mortierella alpina, Pythium insidiosum, or plants such as soybean, oilseed rape, coconut, oil palm, safflower, flax, hemp, castor-oil plant, Calendula, peanut, cacao bean or sunflower, or yeasts such as Saccharomyces cerevisiae, with soybean, flax, oilseed rape, safflower, sunflower, Calendula, Mortierella or Saccharomyces cerevisiae being especially preferred. In principle, suitable host organisms are, in addition to the abovementioned transgenic organisms, also transgenic animals, advantageously nonhuman animals, for example C. elegans.

Further utilizable host cells are detailed in: Goeddel, Gene Expression Technology: Methods in Enzymology 185, Academic Press, San Diego, Calif. (1990).

Expression strains which can be used, for example those with a lower protease activity, are described in: Gottesman, S., Gene Expression Technology: Methods in Enzymology 185, Academic Press, San Diego, Calif. (1990) 119-128.

These include plant cells and certain tissues, organs and parts of plants in all their phenotypic forms such as anthers, fibers, root hairs, stalks, embryos, calli, cotyledons, petioles, harvested material, plant tissue, reproductive tissue and cell cultures which are derived from the actual transgenic plant and/or can be used for giving rise to the transgenic plant.

Transgenic plants which comprise the polyunsaturated fatty acids synthesized in the process according to the invention can advantageously be marketed directly without there being any need for the oils, lipids or fatty acids synthesized to be isolated. Plants for the process according to the invention are listed as meaning intact plants and all plant parts, plant organs or plant parts such as leaf, stem, seeds, root, tubers, anthers, fibers, root hairs, stalks, embryos, calli, cotyledons, petioles, harvested material, plant tissue, reproductive tissue and cell cultures which are derived from the transgenic plant and/or can be used for giving rise to the transgenic plant. In this context, the seed comprises all parts of the seed such as the seed coats, epidermal cells, seed cells, endosperm or embryonic tissue. However, the compounds produced in the process according to the invention can also be isolated from the organisms, advantageously plants, in the form of their oils, fat, lipids and/or free fatty acids. Polyunsaturated fatty acids produced by this process can be obtained by harvesting the organisms, either from the crop in which they grow, or from the field. This can be done via pressing or extraction of the plant parts, preferably the plant seeds. In this context, the oils, fats, lipids and/or free fatty acids can be obtained by what is known as cold-beating or cold-pressing without applying heat by pressing. To allow for greater ease of disruption of the plant parts, specifically the seeds, they are previously comminuted, steamed or roasted. The seeds which have been pretreated in this manner can subsequently be pressed or extracted with solvents such as warm hexane. The solvent is subsequently removed again.

In the case of microorganisms, the latter are, after harvesting, for example extracted directly without further processing steps or else, after disruption, extracted via various methods with which the skilled worker is familiar. In this manner, more than 96% of the compounds produced in the process can be isolated. Thereafter, the resulting products are processed further, i.e. refined. In this process, substances such as the plant mucilages and suspended matter are first removed. What is known as desliming can be effected enzymatically or, for example, chemico-physically by addition of acid such as phosphoric acid. Thereafter, the free fatty acids are removed by treatment with a base, for example sodium hydroxide solution. The resulting product is washed thoroughly with water to remove the alkali remaining in the product and then dried. To remove the pigments remaining in the product, the products are subjected to bleaching, for example using fuller's earth or active charcoal. At the end, the product is deodorized, for example using steam.

The PUFAs or LCPUFAs produced by this process are preferably C₁₈₋, C₂₀-, C₂₂- or C₂₄-fatty acid molecules with at least two double bonds in the fatty acid molecule, preferably three, four, five or six double bonds. These C₁₈-, C₂₀-, C₂₂- or C₂₄-fatty acid molecules can be isolated from the organism in the form of an oil, a lipid or a free fatty acid. Suitable organisms are, for example, those mentioned above. Preferred organisms are transgenic plants.

One embodiment of the invention is therefore oils, lipids or fatty acids or fractions thereof which have been produced by the above-described process, especially preferably oil, lipid or a fatty acid composition comprising PUFAs and being derived from transgenic plants.

A further embodiment according to the invention is the use of the oil, lipid, the fatty acids and/or the fatty acid composition in feedstuffs, foodstuffs, cosmetics or pharmaceuticals.

The term “oil”, “lipid” or “fat” is understood as meaning a fatty acid mixture comprising unsaturated or saturated, preferably esterified, fatty acid(s). The oil, lipid or fat is preferably high in polyunsaturated free or, advantageously, esterified fatty acid(s), in particular linoleic acid, γ-linolenic acid, dihomo-γ-linolenic acid, arachidonic acid, α-linolenic acid, stearidonic acid, eicosatetraenoic acid, eicosapentaenoic acid, docosapentaenoic acid or docosahexaenoic acid. The content of unsaturated esterified fatty acids preferably amounts to approximately 30%, a content of 50% is more preferred, and a content of 60%, 70%, 80% or more is even more preferred. For the analysis, the fatty acid content can, for example, be determined by gas chromatography after converting the fatty acids into the methyl esters by transesterification. The oil, lipid or fat can comprise various other saturated or unsaturated fatty acids, for example calendulic acid, palmitic acid, palmitoleic acid, stearic acid, oleic acid and the like. The content of the various fatty acids in the oil or fat can vary in particular, depending on the starting organism.

The polyunsaturated fatty acids with advantageously at least two double bonds which are produced in the process are, as described above, for example sphingolipids, phosphoglycerides, lipids, glycolipids, phospholipids, monoacylglycerol, diacylglycerol, triacylglycerol or other fatty acid esters.

Starting from the polyunsaturated fatty acids with advantageously at least two double bonds, which acids have been prepared in the process according to the invention, the polyunsaturated fatty acids which are present can be liberated for example via treatment with alkali, for example aqueous KOH or NaOH, or acid hydrolysis, advantageously in the presence of an alcohol such as methanol or ethanol, or via enzymatic cleavage, and isolated via, for example, phase separation and subsequent acidification via, for example, H₂SO₄. The fatty acids can also be liberated directly without the above-described processing step.

After their introduction into an organism, advantageously a plant cell or plant, the nucleic acids used in the process can either be present on a separate plasmid or integrated into the genome of the host cell. In the case of integration into the genome, integration can be random or else be effected by recombination such that the native gene is replaced by the copy introduced, whereby the production of the desired compound by the cell is modulated, or by the use of a gene in trans, so that the gene is linked functionally with a functional expression unit which comprises at least one sequence which ensures the expression of a gene and at least one sequence which ensures the polyadenylation of a functionally transcribed gene. The nucleic acids are advantageously introduced into the organisms via multi-expression cassettes or constructs for multiparallel expression, advantageously into the plants for the multiparallel seed-specific expression of genes.

Mosses and algae are the only known plant systems which produce substantial amounts of polyunsaturated fatty acids such as arachidonic acid (ARA) and/or eicosapentaenoic acid (EPA) and/or docosahexaenoic acid (DHA). Mosses comprise PUFAs in membrane lipids, while algae, organisms which are related to algae and a few fungi also accumulate substantial amounts of PUFAs in the triacylglycerol fraction. This is why nucleic acid molecules are suitable which are isolated from such strains which also accumulate PUFAs in the triacylglycerol fraction, particularly advantageously for the process according to the invention and thus for the modification of the lipid and PUFA production system in a host, in particular plants such as oil crop plants, for example oilseed rape, canola, linseed, hemp, soybeans, sunflowers and borage. They can therefore be used advantageously in the process according to the invention.

To produce the long-chain PUFAs according to the invention, the polyunsaturated C₁₆- or C₁₈-fatty acids must first be desaturated by the enzymatic activity of a desaturase and subsequently be elongated by at least two carbon atoms via an elongase. After one elongation cycle, this enzyme activity gives C₁₈- or C₂₀-fatty acids and after two or three elongation cycles C₂₂- or C₂₄-fatty acids. The activity of the desaturases and elongases used in the process according to the invention preferably leads to C₁₈-, C₂₀-, C₂₂- and/or C₂₄-fatty acids, advantageously with at least two double bonds in the fatty acid molecule, preferably with three, four or five double bonds, especially preferably to give C₂₀- and/or C₂₂-fatty acids with at least two double bonds in the fatty acid molecule, preferably with three, four or five double bonds in the molecule. After a first desaturation and the elongation have taken place, further desaturation steps such as, for example, one in the Δ5 position may take place. Products of the process according to the invention which are especially preferred are dihomo-γ-linolenic acid, arachidonic acid, eicosapentaenoic acid, docosapentaenoic acid and/or docosahexaenoic acid. The C₁₈-fatty acids with at least two double bonds in the fatty acid can be elongated by the enzymatic activity according to the invention in the form of the free fatty acid or in the form of the esters, such as phospholipids, glycolipids, sphingolipids, phosphoglycerides, monoacylglycerol, diacylglycerol or triacylglycerol.

The preferred biosynthesis site of fatty acids, oils, lipids or fats in the plants which are advantageously used is, for example, in general the seed or cell strata of the seed, so that seed-specific expression of the nucleic acids used in the process makes sense. However, it is obvious that the biosynthesis of fatty acids, oils or lipids need not be limited to the seed tissue, but can also take place in a tissue-specific manner in all the other parts of the plant, for example in epidermal cells or in the tubers.

If microorganisms such as yeasts, such as Saccharomyces or Schizosaccharomyces, fungi such as Mortierella, Aspergillus, Phytophtora, Entomophthora, Mucor or Thraustochytrium, algae such as Isochrysis, Phaeodactylum or Crypthecodinium are used as organisms in the process according to the invention, these organisms are advantageously grown in fermentation cultures.

In principle, the polyunsaturated fatty acids produced by the process according to the invention in the organisms used in the process can typically be increased in two different ways. Advantageously, the pool of free polyunsaturated fatty acids and/or the content of the esterified polyunsaturated fatty acids produced via the process can be enlarged. Advantageously, the pool of esterified polyunsaturated fatty acids in the transgenic organisms is enlarged by the process according to the invention.

If microorganisms are used as organisms in the process according to the invention, they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen. The pH of the liquid medium can either be kept constant, that is to say regulated during the culturing period, or not. The cultures can be grown batchwise, semibatchwise or continuously. Nutrients can be provided at the beginning of the fermentation or fed in semicontinuously or continuously. The polyunsaturated fatty acids produced can be isolated from the organisms as described above by processes known to the skilled worker, for example by extraction, distillation, crystallization, if appropriate precipitation with salt, and/or chromatography. To this end, the organisms can advantageously be disrupted beforehand.

If the host organisms are microorganisms, the process according to the invention is advantageously carried out at a temperature of between 0° C. and 95° C., preferably between 10° C. and 85° C., especially preferably between 15° C. and 75° C., very especially preferably between 15° C. and 45° C.

In this process, the pH value is advantageously kept between pH 4 and 12, preferably between pH 6 and 9, especially preferably between pH 7 and 8.

The process according to the invention can be operated batchwise, semibatchwise or continuously. An overview of known cultivation methods can be found in the textbook by Chmiel (Bioprozeβtechnik 1. Einführung in die Bioverfahrenstechnik [Bioprocess technology 1. Introduction to Bioprocess technology] (Gustav Fischer Verlag, Stuttgart, 1991)) or in the textbook by Storhas (Bioreaktoren und periphere Einrichtungen [Bioreactors and peripheral equipment] (Vieweg Verlag, Brunswick/Wiesbaden, 1994)).

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of very good carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The temperature of the culture is normally 20° C. to 45° C. and preferably 25° C. to 40° C. The culture is continued until formation of the desired product is at a maximum. This aim is normally achieved within 10 to 160 hours.

The fermentation broths obtained in this way, in particular those comprising polyunsaturated fatty acids, usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein.

The fatty acids obtained in the process are also suitable as starting material for the chemical synthesis of further products of interest. For example, they can be used in combination with one another or alone for the preparation of pharmaceuticals, foodstuffs, animal feeds or cosmetics.

To introduce the nucleic acids used in the process, the latter are advantageously amplified and ligated in the known manner. Preferably, a procedure following the protocol for Pfu DNA polymerase or a Pfu/Taq DNA polymerase mixture is followed. The primers are selected taking into consideration the sequence to be amplified. The primers should expediently be chosen in such a way that the amplificate comprises the entire codogenic sequence from the start codon to the stop codon. After the amplification, the amplificate is expediently analyzed. For example, a gel-electrophoretic separation can be carried out with regards to quality and quantity. Thereafter, the amplificate can be purified following a standard protocol (for example Qiagen). An aliquot of the purified amplificate is then available for the subsequent cloning step. Suitable cloning vectors are generally known to the skilled worker. These include, in particular, vectors which are capable of replication in microbial systems, that is to say mainly vectors which ensure efficient cloning in yeasts or fungi and which make possible the stable transformation of plants. Those which must be mentioned in particular are various binary and co-integrated vector systems which are suitable for the T-DNA-mediated transformation. Such vector systems are, as a rule, characterized in that they comprise at least the vir genes required for the Agrobacterium-mediated transformation and the T-DNA-delimiting sequences (T-DNA border). These vector systems preferably also comprise further cis-regulatory regions such as promoters and terminators and/or selection markers, by means of which suitably transformed organisms can be identified. While in the case of cointegrated vector systems vir genes and T-DNA sequences are arranged on the same vector, binary systems are based on at least two vectors, one of which bears vir genes, but no T-DNA, while a second one bears T-DNA, but no vir gene. Owing to this fact, the last-mentioned vectors are relatively small, easy to manipulate and to replicate both in E. coli and in Agrobacterium. These binary vectors include vectors from the series pBIB-HYG, pPZP, pBecks, pGreen. In accordance with the invention, Bin19, pBI101, pBinAR, pGPTV and pCAMBIA are used by preference. An overview of binary vectors and their use is found in Hellens et al., Trends in Plant Science (2000) 5, 446-451. In order to prepare the vectors, the vectors can first be linearized with restriction endonuclease(s) and then modified enzymatically in a suitable manner. Thereafter, the vector is purified, and an aliquot is employed for the cloning step. In the cloning step, the enzymatically cleaved and, if appropriate, purified amplificate is cloned using vector fragments which have been prepared in a similar manner, using ligase. In this context, a particular nucleic acid construct, or vector or plasmid construct, can have one or else more than one codogenic gene segment. The codogenic gene segments in these constructs are preferably linked functionally with regulatory sequences. The regulatory sequences include, in particular, plant sequences such as the above-described promoters and terminators. The constructs can advantageously be stably propagated in microorganisms, in particular in Escherichia coli and Agrobacterium tumefaciens, under selective conditions and make possible the transfer of heterologous DNA into plants or microorganisms.

The nucleic acids used in the process, the inventive nucleic acids and nucleic acid constructs, can be introduced into organisms such as microorganisms or advantageously plants, advantageously using cloning vectors, and thus be used in the transformation of plants such as those which are published and cited in: Plant Molecular Biology and Biotechnology (CRC Press, Boca Raton, Fla.), Chapter 6/7, pp. 71-119 (1993); F. F. White, Vectors for Gene Transfer in Higher Plants; in: Transgenic Plants, Vol. 1, Engineering and Utilization, Ed.: Kung and R. Wu, Academic Press, 1993, 15-38; B. Jenes et al., Techniques for Gene Transfer, in: Transgenic Plants, Vol. 1, Engineering and Utilization, Ed.: Kung and R. Wu, Academic Press (1993), 128-143; Potrykus, Annu. Rev. Plant Physiol. Plant Molec. Biol. 42 (1991), 205-225. Thus, the nucleic acids, the inventive nucleic acids and nucleic acid constructs, and/or vectors used in the process can be used for the recombinant modification of a broad spectrum of organisms, advantageously plants, so that the latter become better and/or more efficient PUFA producers.

Nucleic acids which can advantageously be used in the process are derived from bacteria, fungi or plants such as algae or mosses, such as the genera Shewanella, Physcomitrella, Thraustochytrium, Fusarium, Phytophtora, Ceratodon, Isochrysis, Aleurita, Muscarioides, Mortierella, Borago, Phaeodactylum, Crypthecodinium or from nematodes such as Caenorhabditis, specifically from the genera and species Shewanella hanedai, Physcomitrella patens, Phytophtora infestans, Fusarium graminaeum, Cryptocodinium cohnii, Ceratodon purpureus, Isochrysis galbana, Aleurita farinosa, Muscarioides viallii, Mortierella alpina, Borago officinalis, Phaeodactylum tricornutum, or especially advantageously from Caenorhabditis elegans.

The nucleic acid sequences used in the process are advantageously introduced into an expression cassette which makes possible the expression of the nucleic acids in organisms such as microorganisms or plants.

In doing so, the nucleic acid sequences which code for the nucleic acids of the invention, and the nucleic acid sequences which code for acyl co A synthetase used in combination, the desaturases and/or the elongases are linked functionally with one or more regulatory signals, advantageously for enhancing gene expression. These regulatory sequences are intended to make possible the specific expression of the genes and proteins. Depending on the host organism, this may mean, for example, that the gene is expressed and/or overexpressed only after induction has taken place, or else that it expresses and/or overexpresses immediately. For example, these regulatory sequences take the form of sequences to which inductors or repressors bind, thus controlling the expression of the nucleic acid. In addition to these novel regulatory sequences, or instead of these sequences, the natural regulation of these sequences may still be present before the actual structural genes and, if appropriate, may have been genetically modified in such a way that natural regulation has been eliminated and expression of the genes has been enhanced. However, the expression cassette (=expression construct=gene construct) can also be simpler in construction, that is to say no additional regulatory signals have been inserted before the nucleic acid sequence or its derivatives, and the natural promoter together with its regulation has not been removed. Instead, the natural regulatory sequence has been mutated in such a way that regulation no longer takes place and/or gene expression is enhanced. These modified promoters can also be positioned on their own before the natural gene in the form of part-sequences (=promoter with parts of the nucleic acid sequences of the invention) in order to enhance the activity. Moreover, the gene construct may advantageously also comprise one or more of what are known as enhancer sequences in functional linkage with the promoter, which make possible an enhanced expression of the nucleic acid sequence. Additional advantageous sequences, such as further regulatory elements or terminators, may also be inserted at the 3′ end of the DNA sequences. The acyl co A synthetase, Δ-4-desaturase, Δ5-desaturase, Δ-6-desaturase and/or Δ-8-desaturase genes and/or Δ-5-elongase, Δ-6-elongase and/or Δ-9-elongase genes, or other genes involved in fatty acid biosynthesis, may be present in one or more copies in the expression cassette (=gene construct). Preferably, only one copy of the gene is present in each expression cassette. This gene construct or the gene constructs can be expressed together in the host organism. In this context, the gene construct(s) can be inserted in one or more vectors and be present in the cell in free form, or else be inserted in the genome. It is advantageous for the insertion of further genes in the host genome when the genes to be expressed are present together in one gene construct.

In this context, the regulatory sequences or factors can, as described above, preferably have a positive effect on the gene expression of the genes introduced, thus enhancing it. Thus, an enhancement of the regulatory elements, advantageously at the transcriptional level, may take place by using strong transcription signals such as promoters and/or enhancers. In addition, however, enhanced translation is also possible, for example by improving the stability of the mRNA.

Advantageous regulatory sequences for the novel process are present for example in promoters such as the cos, tac, trp, tet, trp-tet, lpp, lac, lpp-lac, lacIq, T7, T5, T3, gal, trc, ara, SP6, λ-PR or λ-PL promoter and are advantageously employed in Gram-negative bacteria. Further advantageous regulatory sequences are, for example, present in the Gram-positive promoters amy and SPO2, in the yeast or fungal promoters ADC1, MFα, AC, P-60, CYC1, GAPDH, TEF, rp28, ADH or in the plant promoters CaMV/35S [Franck et al., Cell 21 (1980) 285-294], PRP1 [Ward et al., Plant. Mol. Biol. 22 (1993)], SSU, OCS, lib4, usp, STLS1, B33, nos or in the ubiquitin or phaseolin promoter. Advantageous in this context are also inducible promoters, such as the promoters described in EP-A-0 388 186 (benzylsulfonamide-inducible), Plant J. 2, 1992:397-404 (Gatz et al., tetracycline-inducible), EP-A-0 335 528 (abscisic acid-inducible) or WO 93/21334 (ethanol- or cyclohexenol-inducible). Further suitable plant promoters are the cytosolic FBPase promoter or the ST-LSI promoter of potato (Stockhaus et al., EMBO J. 8, 1989, 2445), the glycine max phosphoribosylpyrophosphate amidotransferase promoter (Genbank Accession No. U87999) or the node-specific promoter described in EP-A-0 249 676. Especially advantageous promoters are promoters which make possible the expression in tissues which are involved in the biosynthesis of fatty acids. Very especially advantageous are seed-specific promoters, such as the USP promoter as described, but also other promoters such as the LeB4, DC3, phaseolin or napin promoter. Further especially advantageous promoters are seed-specific promoters which can be used for monocotyledonous or dicotyledonous plants and which are described in U.S. Pat. No. 5,608,152 (oilseed rape napin promoter), WO 98/45461 (Arabidopsis oleosin promoter), U.S. Pat. No. 5,504,200 (Phaseolus vulgaris phaseolin promoter), WO 91/13980 (Brassica Bce4 promoter), by Baeumlein et al., Plant J., 2, 2, 1992:233-239 (LeB4 promoter from a legume), these promoters being suitable for dicots. Examples of promoters which are suitable for monocots are the barley lpt-2 or lpt-1 promoter (WO 95/15389 and WO 95/23230), the barley hordein promoter and other suitable promoters described in WO 99/16890.

In principle, it is possible to use all natural promoters together with their regulatory sequences, such as those mentioned above, for the novel process. It is also possible and advantageous to use synthetic promoters, either in addition or alone, in particular when they mediate seed-specific expression, such as those described in WO 99/16890.

In order to achieve a particularly high PUFA content, especially in transgenic plants, the PUFA biosynthesis genes should advantageously be expressed in oil crops in a seed-specific manner. To this end, seed-specific promoters can be used, or those promoters which are active in the embryo and/or in the endosperm. In principle, seed-specific promoters can be isolated both from dicotyledonous and from monocotyledonous plants. Advantageous preferred promoters are listed hereinbelow: USP (=unknown seed protein) and vicilin (Vicia faba) [Bäumlein et al., Mol. Gen. Genet., 1991, 225(3)], napin (oilseed rape) [U.S. Pat. No. 5,608,152], acyl carrier protein (oilseed rape) [U.S. Pat. No. 5,315,001 and WO 92/18634], oleosin (Arabidopsis thaliana) [WO 98/45461 and WO 93/20216], phaseolin (Phaseolus vulgaris) [U.S. Pat. No. 5,504,200], Bce4 [WO 91/13980], legumes B4 (LegB4 promoter) [Bäumlein et al., Plant J., 2,2, 1992], Lpt2 and lpt1 (barley) [WO 95/15389 and WO 95/23230], seed-specific promoters from rice, maize and wheat [WO 99/16890], Amy32b, Amy 6-6 and aleurain [U.S. Pat. No. 5,677,474], Bce4 (oilseed rape) [U.S. Pat. No. 5,530,149], glycinin (soybean) [EP 571 741], phosphoenol pyruvate carboxylase (soybean) [JP 06/62870], ADR12-2 (soybean) [WO 98/08962], isocitrate lyase (oilseed rape) [U.S. Pat. No. 5,689,040] or α-amylase (barley) [EP 781 849].

Plant gene expression can also be facilitated via a chemically inducible promoter (see review in Gatz 1997, Annu. Rev. Plant Physiol. Plant Mol. Biol., 48:89-108). Chemically inducible promoters are particularly suitable when it is desired that gene expression should take place in a time-specific manner. Examples of such promoters are a salicylic-acid-inducible promoter (WO 95/19443), a tetracycline-inducible promoter, (Gatz et al. (1992) Plant J. 2, 397-404) and an ethanol-inducible promoter.

To ensure the stable integration of the biosynthesis genes into the transgenic plant over a plurality of generations, each of the nucleic acids which code for acyl-CoA synthetase, Δ-4-desaturase, Δ-5-desaturase, Δ-6-desaturase, Δ-8-desaturase and/or Δ-5-elongase, Δ-6-elongase and/or Δ-9-elongase and which are used in the process should be expressed under the control of a separate promoter, preferably a promoter which differs from the other promoters, since repeating sequence motifs can lead to instability of the T-DNA, or to recombination events. In this context, the expression cassette is advantageously constructed in such a way that a promoter is followed by a suitable cleavage site, advantageously in a poly-linker, for insertion of the nucleic acid to be expressed and, if appropriate, a terminator is positioned behind the poly-linker. This sequence is repeated several times, preferably three, four or five times, so that up to five genes can be combined in one construct and introduced into the transgenic plant in order to be expressed. Advantageously, the sequence is repeated the promoter via the suitable cleavage site, for example in the poly-linker. Advantageously, each nucleic acid sequence has its own promoter and, if appropriate, its own terminator. However, it is also possible to insert a plurality of nucleic acid sequences behind a promoter and, if appropriate, before a terminator. Here, the insertion site, or the sequence, of the inserted nucleic acids in the expression cassette is not of critical importance, that is to say a nucleic acid sequence can be inserted at the first or last position in the cassette without its expression being substantially influenced thereby. Advantageously, different promoters such as, for example, the USP, LegB4 or DC3 promoter, and different terminators can be used in the expression cassette. However, it is also possible to use only one type of promoter in the cassette. This, however, may lead to undesired recombination events.

As described above, the transcription of the genes which have been introduced should advantageously be terminated by suitable terminators at the 3′ end of the biosynthesis genes which have been introduced (behind the stop codon). An example of a sequence which can be used in this context is the OCS1 terminator. As is the case with the promoters, different terminator sequences should be used for each gene.

As described above, the gene construct can also comprise further genes to be introduced into the organisms. It is possible and advantageous to introduce into the host organisms, and to express therein, regulatory genes such as genes for inductors, repressors or enzymes which, owing to their enzyme activity, engage in the regulation of one or more genes of a biosynthetic pathway. These genes can be of heterologous or of homologous origin. Moreover, further biosynthesis genes of the fatty acid or lipid metabolism can advantageously be present in the nucleic acid construct, or gene construct; however, these genes can also be positioned on one or further nucleic acid constructs. Biosynthesis genes of the fatty acid or lipid metabolism which are advantageously used are a gene selected from the group consisting of acyl-CoA dehydrogenase(s), acyl-ACP [=acyl carrier protein] desaturase(s), acyl-ACP thioesterase(s), fatty acid acyltransferase(s), acyl-CoA:lysophospholipid acyltransferase(s), fatty acid synthase(s), fatty acid hydroxylase(s), acetyl-coenzyme A carboxylase(s), acyl-coenzyme A oxidase(s), fatty acid desaturase(s), fatty acid acetylenases, lipoxygenase(s), triacylglycerol lipase(s), alleneoxide synthase(s), hydroperoxide lyase(s) or fatty acid elongase(s) or combinations thereof. Especially advantageous nucleic acid sequences in combination with the nucleic acid of the invention are biosynthesis genes of the fatty acid or lipid metabolism selected from the group consisting of acyl-CoA:lysophospholipid acyltransferase, Δ-4-desaturase, Δ-5-desaturase, Δ-6-desaturase, Δ-8-desaturase, Δ-9-desaturase, Δ-12-desaturase, Δ-5-elongase, Δ-6-elongase or Δ-9-elongase.

In this context, the abovementioned nucleic acids and genes can be cloned into expression cassettes of the invention in combination with other elongases and desaturases and used for transforming plants with the aid of Agrobacterium.

Here, the regulatory sequences or factors can, as described above, preferably have a positive effect on, and thus enhance, the expression of the genes which have been introduced. Thus, enhancement of the regulatory elements can advantageously take place at the transcriptional level by using strong transcription signals such as promoters and/or enhancers. However, an enhanced translation is also possible, for example by improving the stability of the mRNA. In principle, the expression cassettes can be used directly for introduction into the plant or else be introduced into a vector.

These advantageous vectors, preferably expression vectors, comprise the nucleic acids which code for lysophosphatidic acid acyltransferases, glycerol-3-phosphate acyltransferases, diacylglycerol acyltransferases or lecithin cholesterol acyltransferases and which are used in the process, or else a nucleic acid construct which comprises the nucleic acid used either alone or in combination with further biosynthesis genes of the fatty acid or lipid metabolism such as the acyl-CoA:lysophospholipid acyltransferases, Δ-4-desaturase, Δ-5-desaturase, Δ-6-desaturase, Δ-8-desaturase, Δ-9-desaturase, Δ-12-desaturase, Δ-5-elongase, Δ-6-elongase and/or Δ-9-elongase. As used in the present context, the term “vector” refers to a nucleic acid molecule which is capable of transporting another nucleic acid to which it is bound. One type of vector is a “plasmid”, a circular double-stranded DNA loop into which additional DNA segments can be ligated. A further type of vector is a viral vector, it being possible for additional DNA segments to be ligated into the viral genome. Certain vectors are capable of autonomous replication in a host cell into which they have been introduced (for example bacterial vectors with bacterial replication origin). Other vectors are advantageously integrated into the genome of a host cell when they are introduced into the host cell, and thus replicate together with the host genome. Moreover, certain vectors can govern the expression of genes with which they are in functional linkage. These vectors are referred to in the present context as “expression vectors”. Usually, expression vectors which are suitable for DNA recombination techniques take the form of plasmids. In the present description, “plasmid” and “vector” can be used exchangeably since the plasmid is the form of vector which is most frequently used. However, the invention is intended to comprise these other forms of expression vectors, such as viral vectors, which exert similar functions. Furthermore, the term “vector” is also intended to comprise other vectors with which the skilled worker is familiar, such as phages, viruses such as SV40, CMV, TMV, transposons, IS elements, phasmids, phagemids, cosmids, linear or circular DNA.

The recombinant expression vectors advantageously used in the process comprise the nucleic acids described below or the above-described gene construct in a form which is suitable for expressing the nucleic acids used in a host cell, which means that the recombinant expression vectors comprise one or more regulatory sequences, selected on the basis of the host cells to be used for the expression, which regulatory sequence(s) is/are linked functionally with the nucleic acid sequence to be expressed. In a recombinant expression vector, “linked functionally” means that the nucleotide sequence of interest is bound to the regulatory sequence(s) in such a way that the expression of the nucleotide sequence is possible and they are bound to each other in such a way that both sequences carry out the predicted function which is ascribed to the sequence (for example in an in-vitro transcription/translation system, or in a host cell if the vector is introduced into the host cell). The term “regulatory sequence” is intended to comprise promoters, enhancers and other expression control elements (for example polyadenylation signals). These regulatory sequences are described, for example, in Goeddel: Gene Expression Technology: Methods in Enzymology 185, Academic Press, San Diego, Calif. (1990), or see: Gruber and Crosby, in: Methods in Plant Molecular Biology and Biotechnology, CRC Press, Boca Raton, Fla., Ed.: Glick and Thompson, Chapter 7, 89-108, including the references cited therein. Regulatory sequences comprise those which govern the constitutive expression of a nucleotide sequence in many types of host cell and those which govern the direct expression of the nucleotide sequence only in specific host cells under specific conditions. The skilled worker knows that the design of the expression vector can depend on factors such as the choice of host cell to be transformed, the expression level of the desired protein and the like.

The recombinant expression vectors used can be designed for the expression of the nucleic acid of the invention alone or in combination with other nucleic acid encoding fatty acid synthesis enzymes, for example, lysophosphatidic acid acyltransferases, glycerol-3-phosphate acyltransferases, diacylglycerol acyltransferases or lecithin cholesterol acyltransferases, acyl-CoA:lysophospholipid acyltransferases, desaturases and elongases in prokaryotic or eukaryotic cells. This is advantageous since intermediate steps of the vector construction are frequently carried out in microorganisms for the sake of simplicity. For example, lysophosphatidic acid acyltransferase, glycerol-3-phosphate acyltransferase, diacylglycerol acyltransferase, lecithin cholesterol acyltransferase, acyl-CoA:lysophospholipid acyltransferase, desaturase and/or elongase genes can be expressed in bacterial cells, insect cells (using Baculovirus expression vectors), yeast and other fungal cells (see Romanos, M. A., et al. (1992) “Foreign gene expression in yeast: a review”, Yeast 8:423-488; van den Hondel, C. A. M. J. J., et al. (1991) “Heterologous gene expression in filamentous fungi”, in: More Gene Manipulations in Fungi, J. W. Bennet & L. L. Lasure, Ed., pp. 396-428: Academic Press: San Diego; and van den Hondel, C. A. M. J. J., & Punt, P. J. (1991) “Gene transfer systems and vector development for filamentous fungi, in: Applied Molecular Genetics of Fungi, Peberdy, J. F., et al., Ed., pp. 1-28, Cambridge University Press: Cambridge), algae (Falciatore et al., 1999, Marine Biotechnology.1, 3:239-251), ciliates of the types: Holotrichia, Peritrichia, Spirotrichia, Suctoria, Tetrahymena, Paramecium, Colpidium, Glaucoma, Platyophrya, Potomacus, Desaturaseudocohnilembus, Euplotes, Engelmaniella and Stylonychia, in particular of the genus Stylonychia lemnae, using vectors in a transformation method as described in WO 98/01572 and, preferably, in cells of multi-celled plants (see Schmidt, R. and Willmitzer, L. (1988) “High efficiency Agrobacterium tumefaciens-mediated transformation of Arabidopsis thaliana leaf and cotyledon explants” Plant Cell Rep.: 583-586; Plant Molecular Biology and Biotechnology, C Press, Boca Raton, Fla., Chapter 6/7, pp. 71-119 (1993); F. F. White, B. Jenes et al., Techniques for Gene Transfer, in: Transgenic Plants, Vol. 1, Engineering and Utilization, Ed.: Kung and R. Wu, Academic Press (1993), 128-43; Potrykus, Annu. Rev. Plant Physiol. Plant Molec. Biol. 42 (1991), 205-225 (and references cited therein)). Suitable host cells are furthermore discussed in Goeddel, Gene Expression Technology: Methods in Enzymology 185, Academic Press, San Diego, Calif. (1990). As an alternative, the recombinant expression vector can be transcribed and translated in vitro, for example using T7-promoter regulatory sequences and T7-polymerase.

In most cases, the expression of proteins in prokaryotes involves the use of vectors comprising constitutive or inducible promoters which govern the expression of fusion or nonfusion proteins. Typical fusion expression vectors are, inter alia, pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), where glutathione S-transferase (GST), maltose-E binding protein and protein A, respectively, is fused with the recombinant target protein.

Examples of suitable inducible nonfusion E. coli expression vectors are, inter alia, pTrc (Amann et al. (1988) Gene 69:301-315) and pET 11d (Studier et al., Gene Expression Technology Methods in Enzymology 185, Academic Press, San Diego, Calif. (1990) 60-89). The target gene expression from the pTrc vector is based on the transcription from a hybrid trp-lac fusion promoter by the host RNA polymerase. The target gene expression from the vector pET 11d is based on the transcription of a T7-gn10-lac fusion promoter, which is mediated by a viral RNA polymerase (T7 gnl), which is coexpressed. This viral polymerase is provided by the host strains BL21 (DE3) or HMS174 (DE3) from a resident λ-prophage which harbors a T7 gn1 gene under the transcriptional control of the lacUV 5 promoter.

Other vectors which are suitable for prokaryotic organisms are known to the skilled worker, these vectors are, for example in E. coli pLG338, pACYC184, the pBR series such as pBR322, the pUC series such as pUC18 or pUC19, the M113 mp series, pKC30, pRep4, pHS1, pHS2, pPLc236, pMBL24, pLG200, pUR290, pIN-III113-B1, λgt11 or pBdCI, in Streptomyces pIJ101, pIJ364, pIJ702 or pIJ361, in Bacillus pUB110, pC194 or pBD214, in Corynebacterium pSA77 or pAJ667.

In a further embodiment, the expression vector is a yeast expression vector. Examples for vectors for expression in the yeast S. cerevisiae comprise pYeDesaturasec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi [J. W. Bennet & L. L. Lasure, Ed., pp. 396-428: Academic Press: San Diego]. Further suitable yeast vectors are, for example, pAG-1, YEp6, YEp13 or pEMBLYe23.

As an alternative, the acyl-CoA synthetase, lysophosphatidic acid acyltransferases, glycerol-3-phosphate acyltransferases, diacylglycerol acyltransferases, lecithin cholesterol acyltransferases, acyl-CoA:lysophospholipid acyltransferases, desaturases and/or elongases can be expressed in insect cells using Baculovirus expression vectors. Baculovirus vectors which are available for the expression of proteins in cultured insect cells (for example Sf9 cells) comprise the pAc series (Smith et al. (1983) Mol. Cell. Biol. 3:2156-2165) and the pVL series (Lucklow and Summers (1989) Virology 170:31-39).

The abovementioned vectors offer only a small overview of suitable vectors which are possible. Further plasmids are known to the skilled worker and are described, for example, in: Cloning Vectors (Ed. Pouwels, P. H., et al., Elsevier, Amsterdam-N.Y.-Oxford, 1985, ISBN 0 444 904018). For further suitable expression systems for prokaryotic and eukaryotic cells, see the Chapters 16 and 17 in Sambrook, J., Fritsch, E. F., and Maniatis, T., Molecular Cloning: A Laboratory Manual, 2nd edition, Cold Spring Harbor Laboratory, Cold Spring Harbor Laboratory Press, Cold Spring Harbor, N.Y., 1989.

In a further embodiment of the process, acyl-CoA synthetase, lysophosphatidic acid acyltransferases, glycerol-3-phosphate acyltransferases, diacylglycerol acyltransferases, lecithin cholesterol acyltransferases, acyl-CoA:lysophospholipid acyltransferases, desaturases and/or elongases can be expressed in single-celled plant cells (such as algae), see Falciatore et al., 1999, Marine Biotechnology 1 (3):239-251 and references cited therein, and in plant cells from higher plants (for example spermatophytes such as arable crops). Examples of plant expression vectors comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, pp. 15-38.

A plant expression cassette preferably comprises regulatory sequences which are capable of governing the expression of genes in plant cells and which are linked functionally so that each sequence can fulfill its function, such as transcriptional termination, for example polyadenylation signals. Preferred polyadenylation signals are those which are derived from Agrobacterium tumefaciens T-DNA, such as gene 3 of the Ti plasmid pTiACH5 (Gielen et al., EMBO J. 3 (1984) 835 et seq.), which is known as octopine synthase, or functional equivalents thereof, but all other terminators which are functionally active in plants are also suitable.

Since plant gene expression is very often not limited to transcriptional levels, a plant expression cassette preferably comprises other sequences which are linked functionally, such as translation enhancers, for example the overdrive sequence, which comprises the tobacco mosaic virus 5′-untranslated leader sequence, which increases the protein/RNA ratio (Gallie et al., 1987, Nucl. Acids Research 15:8693-8711).

As described above, plant gene expression must be linked functionally with a suitable promoter which triggers gene expression with the correct timing or in a cell- or tissue-specific manner. Utilizable promoters are constitutive promoters (Benfey et al., EMBO J. 8 (1989) 2195-2202), such as those which are derived from plant viruses, such as 35S CAMV (Franck et al., Cell 21 (1980) 285-294), 19S CaMV (see also U.S. Pat. No. 5,352,605 and WO 84/02913), or plant promoters, such as the promoter of the small rubisco subunit, which is described in U.S. Pat. No. 4,962,028.

Other preferred sequences for use in functional linkage in plant gene expression cassettes are targeting sequences, which are required for steering the gene product into its corresponding cell compartment (see a review in Kermode, Crit. Rev. Plant Sci. 15, 4 (1996) 285-423 and references cited therein), for example into the vacuole, into the nucleus, all types of plastids, such as amyloplasts, chloroplasts, chromoplasts, the extracellular space, the mitochondria, the endoplasmic reticulum, elaioplasts, peroxisomes and other compartments of plant cells.

As described above, plant gene expression can also be facilitated via a chemically inducible promoter (see review in Gatz 1997, Annu. Rev. Plant Physiol. Plant Mol. Biol., 48:89-108). Chemically inducible promoters are particularly suitable when it is desired that the gene expression takes place in a time-specific manner. Examples of such promoters are a salicylic-acid-inducible promoter (WO 95/19443), a tetracyclin-inducible promoter (Gatz et al. (1992) Plant J. 2, 397-404) and an ethanol-inducible promoter.

Promoters which respond to biotic or abiotic stress conditions are also suitable, for example the pathogen-induced PRP1 gene promoter (Ward et al., Plant. Mol. Biol. 22 (1993) 361-366), the heat-inducible tomato hsp80 promoter (U.S. Pat. No. 5,187,267), the chill-inducible potato alpha-amylase promoter (WO 96/12814) or the wound-inducible pinII promoter (EP-A-0 375 091).

Especially preferred are those promoters which bring about the gene expression in tissues and organs in which the biosynthesis of fatty acids, lipids and oils takes place, in seed cells, such as cells of the endosperm and of the developing embryo. Suitable promoters are the oilseed rape napin gene promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumine B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley lpt2 or lpt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890.

In particular, it may be desired to bring about the multiparallel expression of the acyl-CoA synthetase, lysophosphatidic acid acyltransferases, glycerol-3-phosphate acyltransferases, diacylglycerol acyltransferases or lecithin cholesterol acyltransferases used in the process alone or in combination with acyl-CoA:lysophospholipid acyltransferases, desaturases and/or elongases. Such expression cassettes can be introduced via the simultaneous transformation of a plurality of individual expression constructs or, preferably, by combining a plurality of expression cassettes on one construct. Also, a plurality of vectors can be transformed with in each case a plurality of expression cassettes and then transferred onto the host cell.

Promoters which are likewise especially suitable are those which bring about plastid-specific expression, since plastids constitute the compartment in which the precursors and some end products of lipid biosynthesis are synthesized. Suitable promoters, such as the viral RNA polymerase promoter, are described in WO 95/16783 and WO 97/06250, and the clpP promoter from Arabidopsis, described in WO 99/46394.

Vector DNA can be introduced into prokaryotic or eukaryotic cells via conventional transformation or transfection techniques. The terms “transformation” and “transfection”, conjugation and transduction, as used in the present context, are intended to comprise a multiplicity of methods known in the prior art for the introduction of foreign nucleic acid (for example DNA) into a host cell, including calcium phosphate or calcium chloride coprecipitation, DEAE-dextran-mediated transfection, lipofection, natural competence, chemically mediated transfer, electroporation or particle bombardment. Suitable methods for the transformation or transfection of host cells, including plant cells, can be found in Sambrook et al. (Molecular Cloning: A Laboratory Manual., 2nd ed., Cold Spring Harbor Laboratory, Cold Spring Harbor Laboratory Press, Cold Spring Harbor, N.Y., 1989) and other laboratory textbooks such as Methods in Molecular Biology, 1995, Vol. 44, Agrobacterium protocols, Ed.: Gartland and Davey, Humana Press, Totowa, N.J.

Host cells which are suitable in principle for taking up the nucleic acid according to the invention, the gene product according to the invention or the vector according to the invention are all prokaryotic or eukaryotic organisms. The host organisms which are advantageously used are microorganisms such as fungi or yeasts, or plant cells, preferably plants or parts thereof. Fungi, yeasts or plants are preferably used, especially preferably plants, very especially preferably plants such as oil crop plants, which are high in lipid compounds, such as oilseed rape, evening primrose, hemp, thistle, peanut, canola, linseed, soybean, safflower, sunflower, borage, or plants such as maize, wheat, rye, oats, triticale, rice, barley, cotton, cassaya, pepper, Tagetes, Solanaceae plants such as potato, tobacco, eggplant and tomato, Vicia species, pea, alfalfa, bushy plants (coffee, cacao, tea), Salix species, trees (oil palm, coconut), and perennial grasses and fodder crops. Especially preferred plants according to the invention are oil crop plants such as soybean, peanut, oilseed rape, canola, linseed, hemp, evening primrose, sunflower, safflower, trees (oil palm, coconut).

The abovementioned nucleic acids according to the invention are derived from organisms such as animals, ciliates, fungi, plants such as algae or dinoflagellates which are capable of synthesizing PUFAs.

In an advantageous embodiment, the term “nucleic acid (molecule)” as used in the present context additionally comprises the untranslated sequence at the 3′ and at the 5′ end of the coding gene region: at least 500, preferably 200, especially preferably 100 nucleotides of the sequence upstream of the 5′ end of the coding region and at least 100, preferably 50, especially preferably 20 nucleotides of the sequence downstream of the 3′ end of the coding gene region. An “isolated” nucleic acid molecule is separated from other nucleic acid molecules which are present in the natural source of the nucleic acid. An “isolated” nucleic acid preferably has no sequences which naturally flank the nucleic acid in the genomic DNA of the organism from which the nucleic acid is derived (for example sequences which are located at the 5′ and 3′ ends of the nucleic acid). In various embodiments, the isolated acyl-CoA synthetase, lysophosphatidic acid acyltransferase, glycerol-3-phosphate acyltransferase, diacylglycerol acyltransferase and/or lecithin cholesterol acyltransferase molecule can comprise for example fewer than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived.

The abovementioned nucleic acids and protein molecules with acyl-CoA synthetase lysophosphatidic acid acyltransferase, glycerol-3-phosphate acyltransferase, diacylglycerol acyltransferase or lecithin cholesterol acyltransferase activity which are involved in the metabolism of lipids and fatty acids, PUFA cofactors and enzymes or in the transport of lipophilic compounds across membranes are used in the process according to the invention for the modulation of the production of PUFAs in transgenic organisms, advantageously in plants, such as maize, wheat, rye, oats, triticale, rice, barley, soybean, peanut, cotton, Linum species such as linseed or flax, Brassica species such as oilseed rape, canola and turnip rape, pepper, sunflower, borage, evening primrose and Tagetes, Solanaceae plants such as potato, tobacco, eggplant and tomato, Vicia species, pea, cassaya, alfalfa, bushy plants (coffee, cacao, tea), Salix species, trees (oil palm, coconut) and perennial grasses and fodder crops, either directly (for example when the overexpression or optimization of a fatty acid biosynthesis protein has a direct effect on the yield, production and/or production efficiency of the fatty acid from modified organisms) and/or can have an indirect effect which nevertheless leads to an enhanced yield, production and/or production efficiency of the PUFAs or a reduction of undesired compounds (for example when the modulation of the metabolism of lipids and fatty acids, cofactors and enzymes leads to modifications of the yield, production and/or production efficiency or the composition of the desired compounds within the cells, which, in turn, can affect the production of one or more fatty acids).

The combination of various precursor molecules and biosynthesis enzymes leads to the production of various fatty acid molecules, which has a decisive effect on lipid composition, since polyunsaturated fatty acids (=PUFAs) are not only incorporated into triacylglycerol but also into membrane lipids.

Lipid synthesis can be divided into two sections: the synthesis of fatty acids and their binding to sn-glycerol-3-phosphate, and the addition or modification of a polar head group. Usual lipids which are used in membranes comprise phospholipids, glycolipids, sphingolipids and phosphoglycerides. Fatty acid synthesis starts with the conversion of acetyl-CoA into malonyl-CoA by acetyl-CoA carboxylase or into acetyl-ACP by acetyl transacylase. After a condensation reaction, these two product molecules together form acetoacetyl-ACP, which is converted via a series of condensation, reduction and dehydratization reactions so that a saturated fatty acid molecule with the desired chain length is obtained. The production of the unsaturated fatty acids from these molecules is catalyzed by specific desaturases, either aerobically by means of molecular oxygen or anaerobically (regarding the fatty acid synthesis in microorganisms, see F. C. Neidhardt et al. (1996) E. coli and Salmonella. ASM Press: Washington, D.C., pp. 612-636 and references cited therein; Lengeler et al. (Ed.) (1999) Biology of Procaryotes. Thieme: Stuttgart, N.Y., and the references therein, and Magnuson, K., et al. (1993) Microbiological Reviews 57:522-542 and the references therein). To undergo the further elongation steps, the resulting phospholipid-bound fatty acids must then be returned from the phospholipids to the fatty acid CoA ester pool. This is made possible by acyl-CoA:lysophospholipid acyltransferases. Moreover, these enzymes are capable of transferring the elongated fatty acids from the CoA esters back to the phospholipids. If appropriate, this reaction sequence can be followed repeatedly.

Examples of precursors for the biosynthesis of PUFAs are oleic acid, linoleic acid and linolenic acid. These C₁₈-carbon fatty acids must be elongated to C₂₀ and C₂₂ in order to obtain fatty acids of the eicosa and docosa chain type. With the aid of the lysophosphatidic acid acyltransferases, glycerol-3-phosphate acyltransferases, diacylglycerol acyltransferases, lecithin cholesterol acyltransferases used in the process, advantageously in combination with acyl-CoA:lysophospholipid acyltransferases, desaturases such as Δ-4-, Δ-5-, Δ-6- and Δ-8-desaturases and/or Δ-5-Δ-6-, Δ-9-elongases, arachidonic acid, eicosapentaenoic acid, docosapentaenoic acid or docosahexaenoic acid and various other long-chain PUFAs can be obtained, extracted and employed in various applications regarding foodstuffs, feedstuffs, cosmetics or pharmaceuticals. Preferably, C₁₈-, C₂₀-, C₂₂- and/or C₂₄-fatty acids with at least two, advantageously at least three, four, five or six, double bonds in the fatty acid molecule can be prepared using the abovementioned enzymes, to give preferably C₂₀-, C₂₂- and/or C₂₄-fatty acids with advantageously three, four or five double bonds in the fatty acid molecule. Desaturation may take place before or after elongation of the fatty acid in question. This is why the products of the desaturase activities and the further desaturation and elongation steps which are possible result in preferred PUFAs with a higher degree of desaturation, including a further elongation from C₂₀- to C₂₂-fatty acids, to fatty acids such as γ-linolenic acid, dihomo-γ-linolenic acid, arachidonic acid, stearidonic acid, eicosatetraenoic acid or eicosapentaenoic acid. Substrates of the lysophosphatidic acyltransferases, glycerol-3-phosphate acyltransferases, diacylglycerol acyltransferases or lecithin cholesterol acyltransferases in the process according to the invention are C₁₈-, C₂₀- or C₂₂-fatty acids such as, for example, linoleic acid, γ-linolenic acid, α-linolenic acid, dihomo-γ-linolenic acid, eicosatetraenoic acid or stearidonic acid. Preferred substrates are linoleic acid, γ-linolenic acid and/or α-linolenic acid, dihomo-γ-linolenic acid, arachidonic acid, eicosatetraenoic acid or eicosapentaenoic acid. The C₁₈-, C₂₀- or C₂₂-fatty acids with at least two double bonds in the fatty acid are obtained in the process according to the invention in the form of the free fatty acid or in the form of their esters, for example in the form of their glycerides.

The term “glyceride” is understood as meaning a glycerol esterified with one, two or three carboxyl radicals (mono-, di- or triglyceride). “Glyceride” is also understood as meaning a mixture of various glycerides. The glyceride or glyceride mixture may comprise further additions, for example free fatty acids, antioxidants, proteins, carbohydrates, vitamins and/or other substances.

For the purposes of the process of the invention, a “glyceride” is furthermore understood as meaning glycerol derivatives. In addition to the above-described fatty acid glycerides, these also include glycerophospholipids and glyceroglycolipids. Preferred examples which may be mentioned in this context are the glycerophospholipids such as lecithin (phosphatidylcholine), cardiolipin, phosphatidylglycerol, phosphatidylserine and alkylacylglycerophospholipids.

Furthermore, fatty acids must subsequently be translocated to various modification sites and incorporated into the triacylglycerol storage lipid. A further important step in lipid synthesis is the transfer of fatty acids to the polar head groups, for example by glycerol fatty acid acyltransferase (see Frentzen, 1998, Lipid, 100(4-5):161-166).

For publications on plant fatty acid biosynthesis and on the desaturation, the lipid metabolism and the membrane transport of lipidic compounds, on beta-oxidation, fatty acid modification and cofactors, triacylglycerol storage and triacylglycerol assembly, including the references therein, see the following papers: Kinney, 1997, Genetic Engineering, Ed.: J K Setlow, 19:149-166; Ohlrogge and Browse, 1995, Plant Cell 7:957-970; Shanklin and Cahoon, 1998, Annu. Rev. Plant Physiol. Plant Mol. Biol. 49:611-641; Voelker, 1996, Genetic Engineering, Ed.: J K Setlow, 18:111-13; Gerhardt, 1992, Prog. Lipid R. 31:397-417; Guhnemain-Schafer & Kindl, 1995, Biochim. Biophys Acta 1256:181-186; Kunau et al., 1995, Prog. Lipid Res. 34:267-342; Stymne et al., 1993, in: Biochemistry and Molecular Biology of Membrane and Storage Lipids of Plants, Ed.: Murata and Somerville, Rockville, American Society of Plant Physiologists, 150-158, Murphy & Ross 1998, Plant Journal. 13(1):1-16.

The PUFAs produced in the process comprise a group of molecules which higher animals are no longer capable of synthesizing and must therefore take up, or which higher animals are no longer capable of synthesizing themselves in sufficient quantity and must therefore take up additional quantities, although they are synthesized readily by other organisms such as bacteria; for example, cats are no longer capable of synthesizing arachidonic acid.

The term “acyl-CoA synthetase, lysophosphatidic acid acyltransferase, glycerol-3-phosphate acyltransferase, diacylglycerol acyltransferase or lecithin cholesterol acyltransferase” comprises for the purposes of the invention proteins which participate in the biosynthesis of fatty acids and their homologs, derivatives and analogs. Phospholipids for the purposes of the invention are understood as meaning phosphatidylcholine, phosphatidylethanolamine, phosphatidylserine, phosphatidylglycerol and/or phosphatidylinositol, advantageously phosphatidylcholine. The terms lysophosphatidic acid acyltransferase, glycerol-3-phosphate acyltransferase, diacylglycerol acyltransferase or lecithin cholesterol acyltransferase nucleic acid sequence(s) comprise nucleic acid sequences which code for a lysophosphatidic acid acyltransferase, glycerol-3-phosphate acyltransferase, diacylglycerol acyltransferase or lecithin cholesterol acyltransferase and part of which may be a coding region and likewise corresponding 5′ and 3′ untranslated sequence regions. The terms production or productivity are known in the art and encompass the concentration of the fermentation product (compounds of the formula I) which is formed within a specific period of time and in a specific fermentation volume (for example kg of product per hour per liter). The term production efficiency comprises the time required for obtaining a specific production quantity (for example the time required by the cell to establish a certain throughput rate of a fine chemical). The term yield or product/carbon yield is known in the art and comprises the efficiency of the conversion of the carbon source into the product (i.e. the fine chemical). This is usually expressed for example as kg of product per kg of carbon source. By increasing the yield or production of the compound, the amount of the molecules obtained of this compound, or of the suitable molecules of this compound obtained in a specific culture quantity over a specified period of time is increased. The terms biosynthesis or biosynthetic pathway are known in the art and comprise the synthesis of a compound, preferably of an organic compound, by a cell from intermediates, for example in a multi-step and strongly regulated process. The terms catabolism or catabolic pathway are known in the art and comprise the cleavage of a compound, preferably of an organic compound, by a cell to give catabolites (in more general terms, smaller or less complex molecules), for example in a multi-step and strongly regulated process. The term metabolism is known in the art and comprises the totality of the biochemical reactions which take place in an organism. The metabolism of a certain compound (for example the metabolism of a fatty acid) thus comprises the totality of the biosynthetic pathways, modification pathways and catabolic pathways of this compound in the cell which relate to this compound.

The content of all of the references, patent applications, patents and published patent applications cited in the present patent application is herewith incorporated by reference.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures:

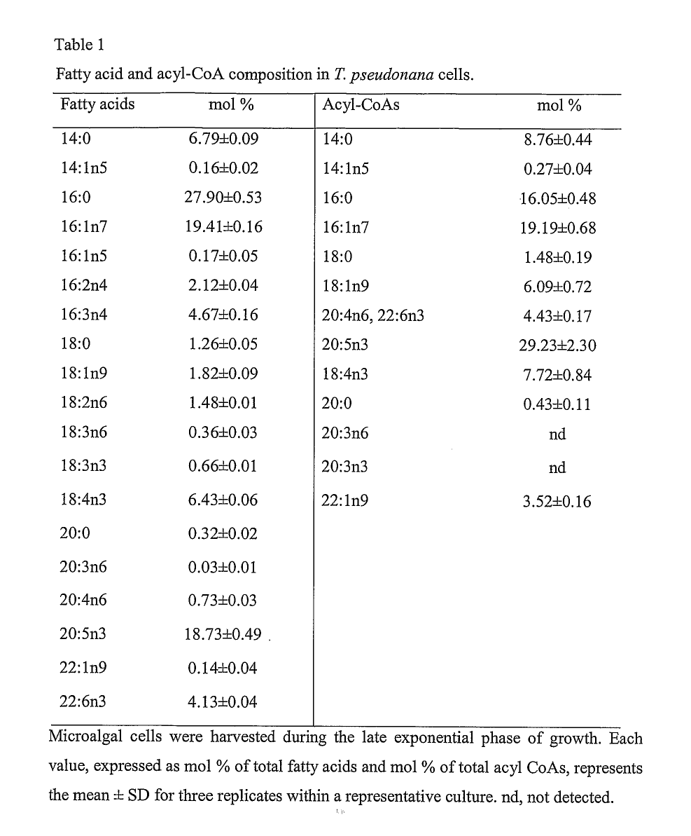

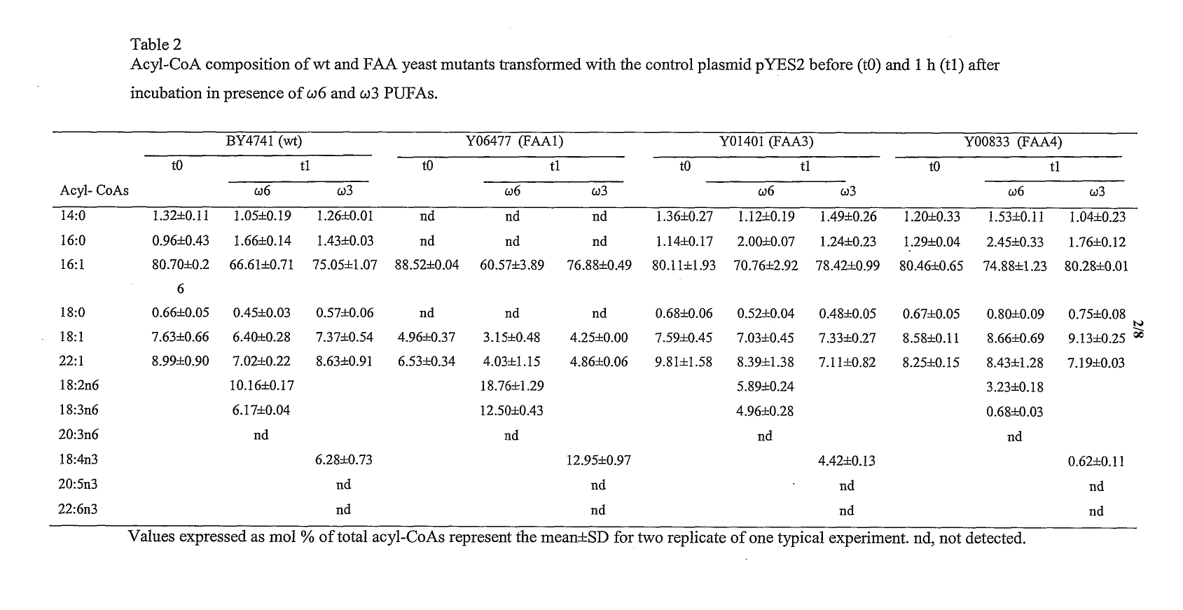

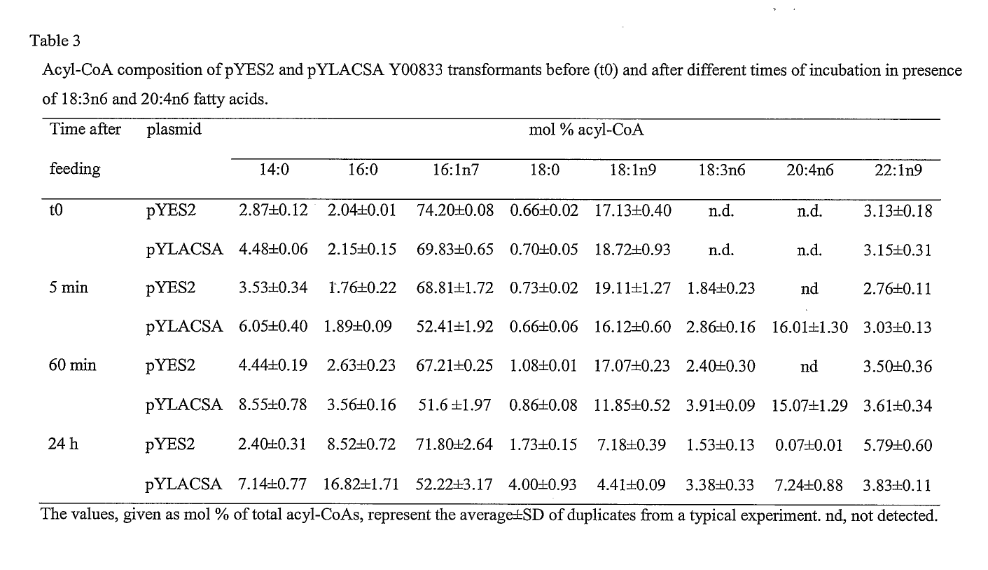

FIG. 1 illustrates RT-PCR expression analysis of TplacsA and TplacsI genes. Thalassiosira cells were harvested at different stages of growth for total RNA extraction and cDNA synthesis. PCR was then performed on undiluted (lane 1) and five-fold serial dilutions (lanes 2-4) of each cDNA using TplacsA and TplacsI specific primer pairs. The 18S rRNA gene was used as a control for cDNA synthesis. Size of the diagnostic fragment for each locus is given between brackets.

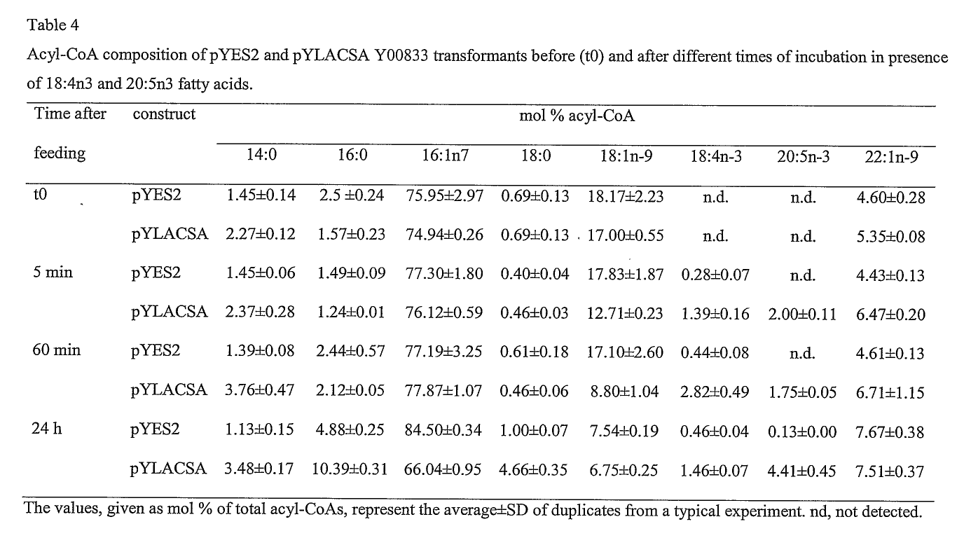

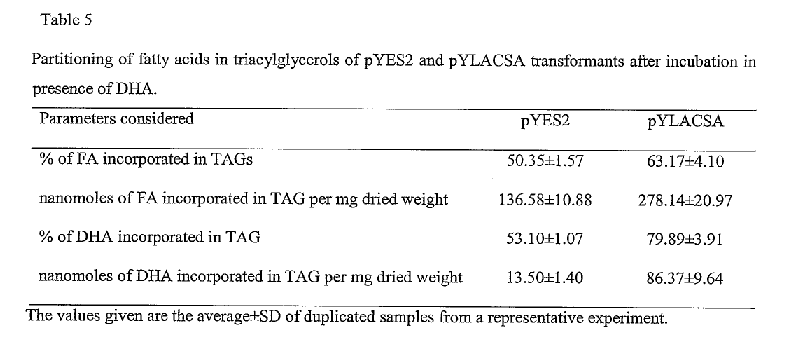

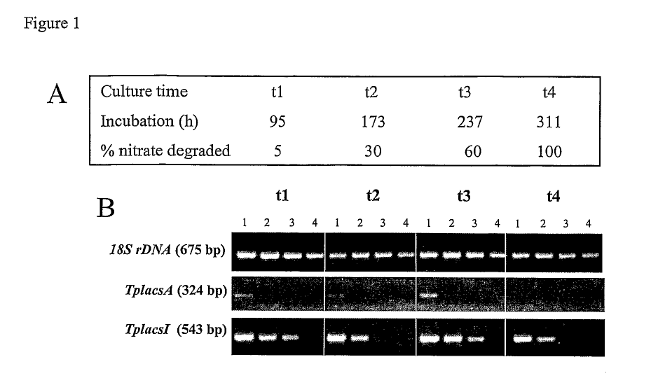

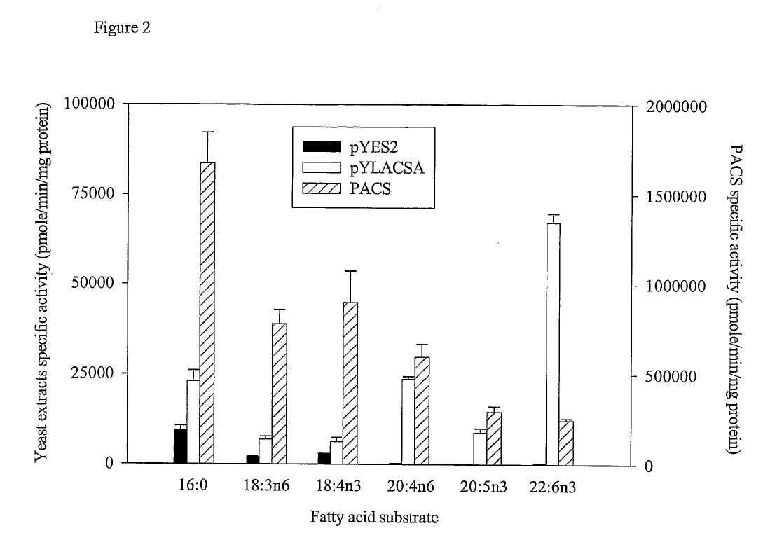

FIG. 2 illustrates LACS enzyme specific activity measurement from cell free lysates of overexpressing Y00833 transformants and from the Pseudoinonas sp. acyl-CoA synthetase (Sigma, PACS). Cell free extracts from yeast containing the plasmid pYES2 (control) and pYLACSA were used as enzymes source in in vitro LACS assay in parallel with the commercially available PACS. Each value represent the average±SD of duplicate acyl-CoA samples during a typical experiment; and

FIG. 3A illustrates the nucleic acid sequence of TpLACSA; and FIG. 3B illustrates the amino acid sequence of TpLACSA.

### MATERIALS AND METHODS

Identification of a set of Genomic DNA Sequences Putatively Encoding Long Chain Acyl-CoA Synthetase

The draft genome of the diatom T. pseudonana has been sequenced to approximately nine times coverage by the whole genome shotgun method. The raw sequence data were downloaded onto a local server from the US Department of Energy Joint Genome Institute (http://wwwjgi.doe.gov/). Batch tblastn searches were carried out using protein sequences of the following 12 known long chain acyl-CoA synthetases as query, including three mammalian proteins: mouse MmLACS4 (BC016416), rat RnLACS4 (D85189), human HsLACS4 (BC034959), and nine Arabidopsis sequences AtLACS1 (AF503751), AtLACS2 (AF503752), AtLACS3 (AF503753), AtLACS4 (AF503754), AtLACS5 (AF503755), AtLACS6 (AF503756), AtLACS7 (AF503757), AtLACS8 (AF503758) and AtLACS9 (AF503759). All non-redundant sequences with an E value less than 0.001 were retrieved and assembled into contigs using the CAP3 sequence assembly programme [12]. The contigs were translated into amino acid sequences in three frames in the orientation indicated by the tblastn result. Eight putative long chain acyl-CoA synthetase gene models were constructed manually based on sequence homology and in-frame GT-AG intron boundaries were identified.

Cultivation of T. pseudonana, RNA Extraction and RT-PCR Analysis

T. pseudonana was cultivated as previously described [13]. Cell density was monitored by counting cells with a haemocytometer. Nitrate concentration was determined periodically during the culture time by measuring the change of the medium absorbance at 220 nm [14].

Total RNA was extracted from cells harvested at different stages of growth with an RNeasy plant mini kit (Qiagen). First strand cDNA was synthesized from three μg of DNAse treated RNA using a Prostar First-strand RT-PCR kit (Stratagene). PCRs with primers pairs specific of putative Thalassiosira long chain acyl-CoA synthetase gene TplacsA was performed using undiluted and five-fold dilutions of cDNAs as followed: the reactions were heated to 95° C. for 5 min followed by 35 cycles at 95° C. for 30 s, 30 s at 55° C. (TplacsA, 18S rRNA) according to the primer pair used and 72° C. for 2 min, then a single step at 72° C. for 10 min. The 18S rRNA gene was used to ensure that the same quantity of cDNA was used for PCR on the different RNA samples. Aliquots of PCR reaction were electrophoresed through a 1% agarose gel.

Heterologous Expression of TplacsA in Yeast

T. pseudonana cDNA was synthesized using the SuperScript™ III RnaseH-Reverse Transcriptase (Invitrogen) and used to amplify the entire TplacsA coding region with primers TpLACSANH 5′-CCCAAGCTTACCATGGCTACGAACAAATGGT-3′ (open reading frame start codon in indicated by bold type; underlined sequence is a HindIII site; italic sequence is an added alanine codon, not present in the original sequence of TplacsA) and TpLACSACE 5′-GCGAATTCTTACAACTTGCTCTGTGGAGA-3′ (ORF stop codon is indicated in bold type; underlined sequence is an EcoRI site). The Expand Long Template PCR System (Roche) was employed to minimize potential PCR errors. The amplified product was first cloned using the TOPO TA cloning kit (Invitrogen) and fidelity of the cloned PCR product was checked by sequencing. Recombinant vector was then restricted with HindIII and EcoRI and cloned in the corresponding sites behind the galactose-inducible GAL1 promoter of pYES2 (Invitrogen) to yield the plasmid pYLACSA. The control vector pYES2 and pYLACSA were then transformed into Saccharomyces cerevisiae by a lithium acetate method, and transformants were selected on minimal medium plates lacking uracil. Host yeast strains were obtained from the Euroscarf yeast deletion strain collection (Frankfurt): wild type BY4741 (MATa; his3Δ1; leu2Δ0, met15Δ0; ura3Δ0) and deletion strains Y06477 (YOR317w::kanMX4, FAA1 mutant), Y01401 (YIL009w::kanMX4, FAA3 mutant), and Y00833 (YMR246w::kanMX4, FAA4 mutant). These three mutated strains are congenic to BY4741.

For the feeding and co-feeding experiments, cultures were grown at 25 or 30° C. in the presence of 2% (w/v) raffinose and 1% (w/v) Tergitol NP-40 (Sigma). Expression of the transgene was induced at OD₆₀₀ₙₘ 0.2-0.3 by supplementing galactose to 2% (w/v). At that time, the appropriate fatty acids were added to a final concentration of 50 μM. For acyl CoA analysis, samples of 3 ml of cells were harvested after 5 min, 1 h and 24 h of incubation at 25° C. For total content and triacylglycerol fatty acids analysis, cells (1.5 ml by sample) were harvested after four days of incubation at 30° C.

Enzyme Overproduction in Yeast and Acyl-CoA Synthetase Assays

Cells were grown overnight in minimum medium lacking uracil containing 2% raffinose and 2% galactose. Following growth, cells were harvested by centrifugation, and resuspended in 100 mM MOPS, pH 7.5, 0.4 mM EDTA, 5 mM 2-mercaptoethanol, 10% glycerol, 0.01% triton X-100 and Protease inhibitor mix (Sigma). This suspension was then transferred in 2 ml Eppendorf tubes containing 500 μl of acid-washed glass beads (425-600 micron, Sigma) and cells lysed by bead-milling for 1 min, five times. Samples were clarified by centrifugation and supernatants used to assess acyl-CoA activities. Protein concentration in these enzyme extracts was determined using the Bradford assay and bovine serum albumin as a standard [15].

Acyl-CoA synthetase activities were determined in yeast cell-free lysates following a protocol adapted from a method based on the use of the Pseudomonas sp. acyl-CoA synthetase (PACS, Sigma) to enzymatically synthesise acyl-CoAs from free fatty acids, ATP, and free CoA [16]. Twenty nanomoles of total free fatty acids were dried down in a 1.5 ml Eppendorf tube. The assay mixture contained 100 mM MOPS pH 7.5, 10 mM MgCl₂, 10 mM ATP, 1 mM dithiothreitol, 0.1% Triton X-100, and 5 mM CoA was added to the tubes and sonicated for 5 min. The reaction was initiated by adding two μl of Pseudomonas sp. enzyme (Sigma) or the same volume of yeast protein extract in tubes placed in a sonicating bath, and incubation was carried out at 25° C. for 25 min. Tubes were sonicated for 5 min and 10 min after starting the assay. The reaction was stopped by addition of 100 μl of 9:2 methanol:chloroform (v/v), 2 μl of saturated (NH₄)₂SO₄, 10 μl of internal standard (17:0-CoA, stock solution at 0.12 mM) and vortexing. After spinning down 5 min at 18,000 g to precipitate proteins, 5 μl of supernatant was transferred to a tapered vial, dried, and 1 ml of chloroacetaldehyde derivitizing buffer was added. Samples were then heated in an oven at 85° C. for 20 min and 20 μl were used for acyl-CoA determination as described below.

Fatty Acid and Acyl-CoA Analyses

Yeast and algal cells were harvested by centrifugation. Fatty acid and acyl-CoA extraction and measurement were carried out from the same pellet as reported previously [17,18].

For triacylglycerol analysis, yeast cells were harvested by centrifugation in pre-weighed tubes, washed with distilled water, and centrifuged overnight in a speedy-vacuum blotter to determine the dry weight. The day after, the pellet was rehydrated with 10 μl of water, then 10 μl of tripentadecanoin (5 mg/ml) and 700 μl of 2:1 chloroform:methanol (v/v) were added. Cells were transferred to a 1.5 ml Eppendorf tube containing 300 μl acid-washed glass beads (425-600 micron, Sigma) and lysed by bead milling twice for 3 min. Extraction and measurement of total fatty acids and triacylglycerol fatty acids was conducted as described previously [11].

### EXAMPLE 1

Fatty Acid and Acyl-CoA Composition of T. pseudonana

Fatty acid profiling of Thalassiosira cells showed that palmitic acid (16:0), palmitoleic acid (16:1n7) and EPA were the most abundant FA in algal cells (Table 1). Only a low percentage of ω6 C20 PUFAs were measured, in contrast with the significant amounts of ω3 stearidonic acid (STA, 18:4n3) and DHA, indicating that the ω3 pathway is the most active in these diatom cells. The acyl CoA profile followed that of FAs in that palmitic, palmitoleic and EPA CoA were the most abundant with the latter representing almost 30% of the acyl CoA pool. This high level of EPA-CoA could potentially act as an intermediate in the synthesis of DHA through elongation to 22:5n3 and desaturation to 22:6n3.

### EXAMPLE 2

Identification of Putative LACS genes in T. pseudonana

TplacsA was found to be full-length in the current sequence data and was predicted to contain two introns. In order to monitor the transcription of TplacsA in Thalassiosira cells, temporal expression analysis was carried out by RT-PCR. FIG. 1 showed that TplacsA was expressed throughout cell cultivation. Amplification and sequencing of the TplacsA ORF from algal cDNA shows that it was 2025 bp long and encodes a protein of 674 amino acids. Alignment of this ORF with the corresponding genomic DNA sequence confirmed the presence of two introns of 96 bp and 88 bp respectively in the second half of the sequence. Comparison of TpLACSA amino acid sequence with functionally characterized LACS showed that the algal enzyme exhibits 35-40% identity with both plant and mammalian LACS, with high homology in the region containing a putative AMP-binding domain. Our further studies focused on the functional characterization of TplacsA.

### EXAMPLE 3

Evaluation of Fatty Acid Activation Deletion Mutants of Saccharoinyces Cereviseae

In order to identify an optimal S. cereviseae strain for the functional characterization of TplacsA several Fatty Acid Activation (FAA) deletion mutants from the Euroscarf collection were tested. Proteins encoded by the genes FAA1 and FAA4 have been shown to be the primary enzymes involved in activation of imported C12 to C18 FAs, while FAA3 was found to be most active towards fatty acids longer than C18 [8]. Wild type strain BY4741 and deletion strains Y06477, Y01401 and Y00833 were transformed with the empty vector control, pYES2, and incubated simultaneously in the presence of three ω6 (18:2n6, 18:3n6, 20:3n6) or three ω3 (18:4n3, 20:5n3, 22:6n3) PUFAs. Table 2 shows the acyl-CoA composition after 1 h incubation at 25° C. in these different strains. Surprisingly, neither C20 nor C22 PUFA-CoAs could be detected in wild type or FAA mutants, suggesting that the cells were not able to produce the corresponding acyl-CoAs during this short time of incubation. However, the fatty acids used as substrates were incorporated by the four strains since FA profiling showed they were present in washed yeast cells (data not shown). No 14:0, 16:0 nor 18:0-CoAs could be detected in Y06477 cells suggesting that the FAA1 gene product is involved in the activation of the corresponding saturated fatty acids. Similar percentages of 18:3n6 and 18:4n3 CoAs were measured in wild type cells, but their amounts were lower than the values determined for 18:2n6. In all the different lines, a higher 18:2n6 CoA percentage suggested that this FA is efficiently incorporated and/or activated in yeast cells. Compared with the wild type cells, Y00833 exhibited the lowest content of acyl CoAs synthesised from exogenously fed unsaturated eighteen carbon CoAs. This suggests that the FAA4 gene product plays a major role in the activation of unsaturated fatty acids in yeast cells. Y00833 was selected as a useful line for heterologous expression studies aimed at identification of genes encoding PUFA synthetase activity on the basis that it has much lower background acyl CoA synthetase activity with PUFAs, and zero activity with 20:5n3 and 22:6n3.

### EXAMPLE 4

Heterologous Expression of TplacsA in S. cereviseae FAA Deletion Strain Y00833

In order to establish the function of the TpLACSA protein, the full length TplacsA cDNA was cloned behind the galactose-inducible GAL1 promoter of pYES2 to generate the plasmid pYLACSA. The results of incubation experiments conducted separately in the presence of the ω6 18:3n6 and 20:4n6, and ω3 18:4n3 and 20:5n3 FAs are presented in Tables 3 and 4 respectively. After 5 min of incubation, C18 PUFA-CoAs were found in both empty vector control pYES2 and pYLACSA Y00833 transformants, with a higher percentage in the latter. No C20 PUFA-CoAs were detected in the empty vector control Y00833, in contrast with Y00833 containing the TplacsA gene. ARA-CoA was the most abundant of the PUFA-CoAs measured in pYLACSA transformants, peaking in concentration after 5 minutes incubation and then falling to approximately half this initial concentration over the following 24 hours. The four exogenously fed fatty acids accumulated in the cells and did not follow the temporal variation exhibited by the corresponding acyl-CoAs (data not shown). C20 PUFA-CoAs were not detected in the empty vector controls after 60 minutes but were detected 24 hours after feeding. C18 ω3 and ω6 FAs followed a similar pattern of accumulation as ARA-CoA in pYLACSA transformants with values increasing during the first hour of incubation and then decreasing after 24 hours. In contrast, EPA-CoA increased throughout the duration of the experiment. TpLACSA also led to a two-fold increase in the endogenous saturated 14:0, 16:0 and 18:0-CoAs, while 16:1 and 18:1-CoAs decreased, and 22.1-CoA was only slightly changed.

### EXAMPLE 5

Measurement of Acyl-CoA Synthetase Activities by in vitro Assay

In order to determine the substrate specificity of TpLACSA directly, several fatty acids were tested using an assay adapted to measure the enzymatic production of acyl-CoA in the presence of free fatty acids, ATP and free CoA. A commercially available acyl-CoA synthetase from Pseudomonas sp. that utilizes a broad range of fatty acid substrates was included as a positive control. Results shown in FIG. 2 confirm the broad specificity of this enzyme. Comparison of specific activities determined in the extract obtained from the pYES2 and the pYLACSA Y00833 transformants showed that TpLACSA is very active on C20 and C22 PUFAs. Effectively, activities were 62 to 222-fold higher for 20:4n6, 20:5n3 and 22:6n3 FAs in the TpLACSA extract compared to the empty vector control, while values in the assays conducted in the presence of palmitic acid or C18 PUFAs only increased by a factor of 2-3. Production of acyl-CoAs in the presence of ARA, EPA and DHA free fatty acids were barely detectable in the pYES2 yeast extract.

### EXAMPLE 6

DHA Storage in Yeast Expressing TplacsA

In order to establish if the expression of the TplacsA gene might result in an increased quantity of 22:6n3 (DHA) stored in yeast storage lipids, total and TAG fatty acids were extracted from pYES2 and pYLACSA Y00833 transformants after four days incubation at 30° C. in the presence of DHA. Table 5 shows that Y00833 containing the TplacsA gene showed approximately six times the amount of DHA and an associated doubling of total FAs in TAG on a dry weight basis compared to the empty vector control. Only a slight increase was observed for endogenous saturated and monounsaturated fatty acids (data not shown).

### REFERENCES

[1] Groot, P. H., Scholte, H. R. and Hulsmann, W. C. (1976) Adv. Lipid Res. 14, 75-126.
[2] Komberg, A. and Pricer, W. E. J. (1953) J. Biol. Chem. 204, 329-343.
[3] Watkins, P. A. (1997) Prog. Lipid. Res. 36, 55-83.
[4] Schnurr, J. A., Shockey, J. M., de Boer, G.-J. and Browse, J. A. (2002) Plant Physiol. 129, 1700-1709.
[5] Shockey, J. M., Fulda, M. S, and Browse, J. A. (2002) Plant Physiology 129, 1710-1722.
[6] Ohlrogge, J. B., Browse, J. and Somerville C. R. (1991) Biochim. Biophys. Acta 1082, 1-26.
[7] Black, P. N., DiRusso, C. C., Metzger, A. K. and Heimert, T. L. (1992) J. Biol. Chem. 267, 25513-25520.
[8] Faergeman, N. J., Black, P. N., Zhao, X. D., Knudsen, J. and DiRusso, C. C. (2001) J. Biol. Chem. 276, 37051-37059.
[9] Mashek, D. G., Bornfeldt, K. E., Coleman, R. A., Berger, J., Bernlohr, D. A., Black, P., DiRusso, C. C., Farber, S. A., Guo, W., Hashimoto, N., Khodiyar, V., Kuypers, F. A., Maltais, L. J., Nebert, D. W., Renieri, A., Schaffer, J. E., Stahl, A., Watkins, P. A., Vasiliou, V. and Yamamoto T. T. (2004) J. Lipid. Res., In Press.
[10] Lauritzen, L., Hansen, H. S., Jorgensen, M. H. and Michaelsen, K. F. (2001) Prog. Lipid. Res. 40, 1-94.
[11] Tonon, T., Harvey, D., Larson, T. R. and Graham, I. A. (2002) Phytochemistry 61, 15-24.
[12] Huang, X. and Madan, A. (1999) Genome Res.9, 868-877.
[13] Tonon, T., Harvey, D., Qing, R., Li, Y., Larson, T. R. and Graham I. A. (2004) FEBS Lett. 563, 28-34.
[14] Collos, Y., Momet, F., Sciandra, A., Waser, N., Larson, A. and Harrison, P. J. (1999) J. Appl. Phycol. 11, 179-184.
[15] Bradford, M. M. (1976). Anal. Biochem. 72, 248-254.
[16] Taylor, D. C., Weber, N., Hogge, L. R. and Underhill, E. W. (1990) Anal. Biochem. 184, 311-316.
[17] Larson, T. R., Edgell, T., Byrne, J., Dehesh, K. and Graham, I. A. (2002) Plant J 32, 519-527.
[18] Larson, T. R. and Graham, I. A. (2001) Plant J. 25, 115-125.
[19] Meyer, A., Kirsch, H., Domergue, F., Abbadi, A., Sperling, P., Bauer, J., Cirpus, P., Zanl, T. K., Moreau, H., Roscoe, T. J., Zahringer, U. and Heinz, E. (2004) J. Lipid. Res., In press.
[20] Pereira, S. L., Leonard, A. E., Huang, Y.-S., Chuang, L.-T. and Mukerji, P. (2004) Biochem. J., In press.
[21] Knoll, L. J., Johnson, D. R. and Gordon, J. I. (1994) J. Biol. Chem. 269, 16348-16356.
[22] Domergue, F., Abbadi, A., Ott, C., Zank, T. K., Zähringer, U. and Heinz, E. (2003) J. Biol. Chem. 278, 35115-35126.
[23] Qi, B., Fraser, T., Mugford, S., Dobson, G., Sayanova, O., Butler, J., Napier, J. A., Stobart, A. K. and Lazarus, C. M. (2004) Nat. Biotechnol. 22, 739-745.

1. A transgenic cell, comprising:
a nucleic acid molecule comprising the nucleic acid sequence represented in SEQ ID NQ: 1, or a nucleic acid molecule that hybridizes to SEQ ID NO: 1 under stringent hybridization conditions, wherein said nucleic acid molecule encodes a polypeptide which has acyl-CoA synthetase activity.

2. The cell according to claim 1, wherein said nucleic acid molecule comprises the nucleic acid sequence as represented in SEQ ID NO: 1.

3. The cell according to claim 1 wherein said nucleic acid molecule consists of the nucleic acid sequence as represented in SEQ ID NO: 1.

4. A transgenic cells wherein said cell is adapted to express a nucleic acid molecule that encodes a polypeptide as represented by the amino acid sequence shown in SEQ ID NO: 2, or a variant of SEQ ID NO: 2 modified by addition, deletion or substitution of at least one amino acid residue and wherein said polypeptide, or variant polypeptide, has acyl-CoA synthetase activity.

5. The cell according to claim 4, wherein said modification retains or enhances the acyl-CoA synthetase enzyme activity of said polypeptide.

6. The cell according to any of claim 1, wherein said nucleic acid molecule is isolated from an algal species.

7. The cell according to claim 1, wherein said acyl-coA synthetase activity modifies 20 and/or 22 carbon polyunsaturated fatty acids.

8. A vector comprising the nucleic acid molecule represented in SEQ ID NO: 1.

9. The vector according to claim 8 wherein said nucleic acid molecule is operably linked to a tissue specific promoter.

10. The vector according to claim 9 wherein said promoter is a seed specific promoter.

11. The vector according to claim 9 wherein said promoter is an inducible promoter or a developmentally regulated promoter.

12. The cell according to claim 1, wherein said cell is a eukaryotic cell.

13. The cell according to claim 1, wherein said cell is a prokaryotic cell.

14. The cell according to claim 12 wherein said eukaryotic cell is a plant cell.

15. A seed comprising the plant cell according claim 14.

16. A method of esterification of a long chain fatty acid to coenzyme A to form acyl-CoA, comprising culturing the cell of claim 1.

17. A reaction vessel comprising:
a polypeptide as represented in SEQ ID NO: 2 or a variant of SEQ ID NO: 2 modified by addition, deletion or substitution of at least one amino acid residue and wherein said polypeptide or variant polypeptide, has acyl-CoA synthetase activity;
a long chain fatty acid,
ATP;
and coenzyme A.

18. The vessel according to claim 17 wherein said vessel is a fermentor.

19. The vessel according to claim 17 wherein said polypeptide is expressed by a cell expressing a nucleic acid molecule comprising the nucleic acid sequence represented in SEQ ID NO: 1 or by a cell expressing a nucleic acid molecule that hybridizes to SEQ ID NO: 1 under stringent hybridization conditions, wherein said nucleic acid molecule encodes a polypeptide having acyl-CoA synthetase activity.

20. The vessel according to claim 19 wherein said cell is a eukaryotic cell.

21. The vessel according to claim 20 wherein said cell is a yeast cell.

22. The vessel according to claim 19 wherein said cell is a prokaryotic cell.

23. A process to esterify a long chain fatty acid substrate to coenzyme A to form acyl-CoA comprising:
i) providing the reaction vessel according claim 19; and
ii) growing cells contained in said reaction vessel under conditions which allow the esterification of a long chain fatty acid to acyl-CoA.

24. The process according to claim 23 wherein said long chain fatty acid is selected from the group consisting of: 18:3n6, 20:4n6, 18:4n3, 20:5n3 and 22:6n3.

25. An oil, a lipid, or a fatty acid composition comprising polyunsaturated fatty acids prepared by the process of claim 23.

26. The composition according to claim 25 wherein said composition originates from a transgenic plant.

27. A feed, foodstuff, cosmetic or pharmaceutical comprising of claim 25.

28. A transgenic plant comprising SEQ ID NO: 1 which encodes a polypeptide having acyl-CoA synthetase activity that modifies 20 and/or 22 carbon polyunsaturated fatty acids.
